(19) United States
(12) Patent Application Publication
(10) Pub. No.: US 2010/0150853 A1
(43) Pub. Date: Jun. 17, 2010

(54) Artificially coloring the skin with a carotene compound, a xanthophyll compound and a lipophilic green dye composition

(75) Inventors: Guillaume Cassin, Villebon Sur Yvette (FR); Sayeh Pourhamidi, Neuilly Sur Marne (FR); Irène Elguidj, Neuilly (FR)

Correspondence Address: BUCHANAN, INGERSOLL & ROONEY PC, POST OFFICE BOX 1404, ALEXANDRIA, VA, 22313-1404, US

(73) Assignee: L'OREAL, Paris (FR)

(21) Appl. No.: 12/591,778

(22) Filed: Dec. 1, 2009

Related U.S. Application Data
(60) Provisional application No. 61/193,580, filed on Dec. 8, 2008.

(30) Foreign Application Priority Data
Dec. 1, 2008 (FR) 0858152

(51) Int. Cl. A61K 8/34; A61K 8/35; A61Q 17/04

(52) U.S. Cl. 424/59

(57) ABSTRACT

A method for artificially coloring the skin entails topical application thereon of a composition containing, formulated into a physiologically acceptable medium:
a) at least one compound of the carotene type,
b) at least one compound of the xanthophyll type,
c) at least one lipophilic green dye; the composition advantageously includes a mixture of dyes including:
a) at least one compound of the carotene type,
b) at least astaxanthin,
c) at least one lipophilic green dye.

### CROSS-REFERENCE TO PRIORITY/PROVISIONAL APPLICATIONS

This application claims priority under 35 U.S.C. §119 of FR 0858152, filed Dec. 1, 2008, and under 35 U.S.C. §120 of U.S. Provisional Application No. 61/193,580, Dec. 8, 2008, each hereby expressly incorporated by reference and each assigned to the assignee hereof.

### BACKGROUND OF THE INVENTION

1. Technical Field of the Invention

The present invention relates to a method for artificially coloring the skin, comprising application thereon of a composition comprising, formulated into a physiologically acceptable medium:

a) at least one compound of the carotene type,

b) at least one compound of the xanthophyll type,

c) at least one lipophilic green dye.

The present invention also relates to a novel mixture of dyes, comprising:

a) at least one compound of the carotene type,

b) at least astaxanthin,

c) at least one lipophilic green dye.

The present invention also relates to a composition for artificially coloring the skin, comprising, in a physiologically acceptable medium, at least said novel mixture of dyes.

2. Description of Background and/or Related and/or Prior Art

It is common for individuals with pigmentation marks, or shadows under the eyes, to wish to correct these cutaneous dyschromias and to use, for this purpose, cosmetic or dermatological compositions that can render the complexion uniform. To this end, it is known practice to use covering products for the purpose of making the complexion immediately uniform, which products do indeed hide skin imperfections, but have the major drawback of masking the natural appearance of the skin (mask sensation).

It is also known practice to use products containing interference pigments such as pearlescent agents, which, although being able to hide skin imperfections, have the major drawback of giving the skin a shiny, unnatural appearance.

Finally, it is known practice to use self-tanning products which are based on carbonyl derivatives such as dihydroxyacetone (DHA), which allow, by interaction with the free amine functions of the skin, in particular the amino acids, peptides and proteins of the skin, the formation of colored products. These products, in particular those based on DHA, by giving a tanned, healthy-complexion appearance, allow skin imperfections to be hidden. Unfortunately, this is only possible on light skin. On dark skin, self-tanning agents such as DHA do not make it possible to produce an adequate coloring.

This is because DHA is characterized by an orangey-yellow color which does not allow an effect sufficient to render the complexion uniform on dark skin. For this, it will be necessary to have a molecule that gives the skin a tint characterized by a greater red component than DHA. In addition, another drawback of self-tanning agents such as DHA is the length of time the coloration takes to develop: specifically, several hours (3 to 5 hours in general) are required for the coloration to be revealed.

Thus, need continues to exist for a novel method of artificial non-covering coloration of the skin that can rapidly give the skin a uniform color and an immediate “healthy complexion” effect that can correct cutaneous dyschromias without the drawbacks mentioned previously and without masking the natural appearance of the skin, whatever the light or dark skin type.

### SUMMARY OF THE INVENTION

Following a considerable amount of research in the field of artificial coloring of the skin, it has now been discovered that, by combining a mixture of lipophilic dyes, constituted of a mixture of at least one carotenoid, of at least one compound of the xanthophyll type and of at least one lipophilic green dye, it is possible to produce, on any light or dark skin type, a uniform complexion and an immediately healthy-complexion effect that permits correction of cutaneous dyschromias without masking the natural appearance of the skin.

The present invention thus features a method for artificially coloring the skin, comprising application thereon of a composition which comprises, formulated into a physiologically acceptable medium:

a) at least one carotene,

b) at least one compound of the xanthophyll type,

c) at least one lipophilic green dye.

The present invention also features a mixture of dyes, comprising:

a) at least one carotene,

b) at least astaxanthin,

c) at least one lipophilic green dye.

The present invention also features compositions containing a mixture of dyes, comprising:

a) at least one carotene,

b) at least astaxanthin,

c) at least one lipophilic green dye.

For the purpose of the present invention, the expression “artificial coloring of the skin” will mean a long-lasting, non-covering coloration (i.e., a coloration that does not have a tendency to opacify the skin). Such a long-lasting coloration is thus distinguished from the superficial and temporary coloration provided, for example, by a makeup product.

For the purpose of the present invention, the term “physiologically acceptable medium” will mean a carrier compatible with the skin, the nails, the lips, the eyelashes and the eyebrows, which has a pleasant color, odor and feel, and which does not create any unacceptable discomfort (tingling, tautness, redness) that may dissuade the consumer from using this composition comprising such a carrier.

The term “lipophilic dye” means any cosmetic or dermatological organic dye capable of being completely dissolved in the molecular state in a liquid fatty phase or else being solubilized in colloidal form (for example, in micellar form) in a liquid fatty phase.

For the purpose of the present invention, the term “liquid fatty phase” means a fatty phase which is liquid at ambient temperature (25° C.) and atmospheric pressure (760 mmHg), composed of one or more fatty substances which are liquid at ambient temperature, also known as oils, and which are compatible with one another.

The term “green dye” means any cosmetic or dermatological organic dye capable of absorbing light radiations having a wavelength of from 400 to 500 nm, and those having a wavelength of from 600 to 700 nm.

Other characteristics, aspects and advantages of the present invention will become apparent from the detailed description which follows.

### DETAILED DESCRIPTION OF BEST MODE AND SPECIFIC/PREFERRED EMBODIMENTS OF THE INVENTION

### Mixture of Lipophilic Dyes

Among the carotenes, mention may in particular be made of:

α-carotene,

β-carotene,

lycopene.

Among the carotenes, more particularly useful is β-carotene (CI-40800, CI-75130, Food Orange 5 or Natural Yellow 26). The β-carotene molecule is a chain constituted of eight isoprene units with alternating single and double bonds, and has the formula:

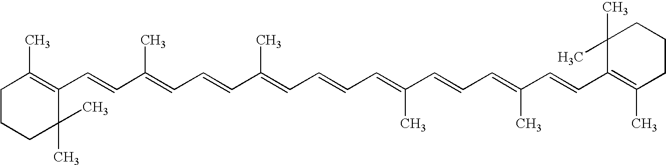

β-carotene is found in certain fruits and vegetables: pepper, carrot, spinach, lettuce, tomato, sweet potato, broccoli, cantaloupe, marrow, apricot. β-carotene can be produced either by extraction, or by synthesis, or biotechnologically. Natural β-carotene comes mainly from alfalfa and red palm oil, and also carrot oil.

According to one particularly preferred embodiment, β-carotene in the form of a dispersion in an oil is employed, such as a dispersion at 30% of β-carotene in sunflower oil, for instance the product marketed under the trademark 409185 Carotene-Dispersion Natural 30% L-OS E-160A produced by LCW—Sensient Cosmetic Technologies, or the dispersion at 30% in maize oil, for instance the product marketed under the trademark 30% Beta Carotene FS (fluid suspension) by DSM Nutritional Products, Inc.

Among the xanthophylls, mention may in particular be made of:

astaxanthin

antheraxanthin

citranaxanthin

cryptoxanthin

canthaxanthin

diatomoxanthin

flavoxanthin

fucoxanthin

lutein

rhodoxanthin

rubixanthin

siphonaxanthin

violaxanthin

zeaxanthin.

Among the xanthophylls, more particularly useful is the astaxanthin of formula:

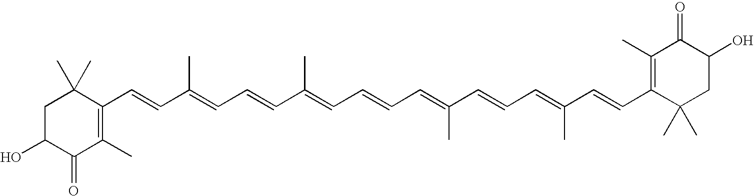

Astaxanthin is, in general, extracted from the alga Haematococcus pluvialis. It belongs to the terpene family, and forms part of the phytochemical compounds. It is present in shell fish (crabs, shrimp, lobster, crayfish, rock lobsters) salmon and red sea bream, and in the feathers of some birds. It may be considered to be the final end-point of a series of hydroxylations and oxidations starting from β-carotene.

According to one particularly preferred embodiment, astaxanthin in the form of a dispersion in an oil is employed, such as a dispersion at 5% of astaxanthin derived from Euphausia Superba in a mixture of caprylic/capric triglycerides, such as the product marketed under the trademark ASTAX-S by Itano Refrigerated Food, or a dispersion at 4.5-7% of asthaxanthin in a mixture of caprylic/capric triglycerides, extracted from the alga Haematococcus pluvialis, such as the product marketed under the trademark Asta Trol-X by Fuji Color or the product marketed under the trademark Bioastin 5% Oleoresin by Cyanotech.

Mention may also be made of the dispersions of asthaxanthin in a mixture of caprylic/capric triglycerides, extracted from the alga Haematococcus pluvialis, such as the commercial products AM Asta-Sod from the company Athena Co Ltd; the commercial products Astaxanthin-5C and Astaxanthin-PC1 marketed by Oryza Oil & Fat Chemical Co.

Among the lipophilic green dyes that can be used according to the invention, exemplary are quinizarin (Ceres Green BB, D&C Green No. 6, CI 61565, 1,4-Di-p-Toluidinoanthraquinone, Green No. 202, Quinizarin Green SS) of formula:

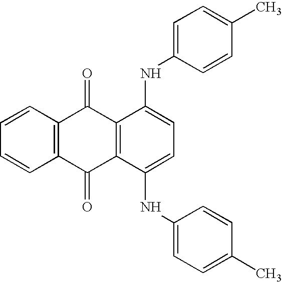

such as the product marketed under the trademark D&C Green 6 K7016 by LCW—Sensient Cosmetic Technologies.

Among the lipophilic green dyes, mention may also be made more preferentially of chlorophylls. Chlorophylls are constituted of four pyrrol rings in a circle in the form of a complex of a divalent cation and also a long-chain alcohol such as phytol. Several forms of chlorophyll, that can be differentiated according to their chemical structure, exist. Chlorophyll a exists in all plants, chlorophyll b is found in higher plants and green algae. Two other variants exist in brown algae and certain cyanobacteria, respectively chlorophylls c and d. The divalent cation(s) present in chlorophylls is (are) selected, in general, from alkali metals such as sodium or potassium, alkaline-earth metals such as calcium or magnesium, and transition metals such as copper or iron, or mixtures thereof.

According to one particularly preferred embodiment, a chlorophyll in the form of a copper complex is employed, and more particularly in the form of a dispersion in an oil such as sunflower oil or grapeseed oil, for instance the commercial products Chlorophylle Liposoluble W 7208, 503509 Copper Chlorophyll 15% L-OS and Chlorophylle Liposoluble W 7208, marketed by LCW—Sensient Cosmetic Technologies.

Depending on the desired effect, the mixture of lipophilic dyes according to the invention is present in the composition for coloring the skin in concentrations ranging preferably from 0.1% to 25%, more preferentially from 0.5% to 15%, and even more preferentially from 0.5% to 10% by weight, relative to the total weight of the composition.

According to one particular embodiment of the invention, the mixture of lipophilic dyes may be used in encapsulated form.

Preferably, the mixture of lipophilic dyes comprises from 1% to 10% by weight of active material of lipophilic green dye, relative to the total weight of the mixture of lipophilic dyes.

Preferably, the mixture of lipophilic dyes is constituted of:

a) from 70% to 90% by weight of active material of carotene compound;

b) from 1% to 20% by weight of active material of xanthophyll compound;

c) from 1% to 10% by weight of active material of green dye, the amounts being defined relative to the total weight of the mixture and the sum of the amounts being equal to 100%.

The mixture of dyes, constituted of at least a) a carotene, b) astaxanthin and c) at least one lipophilic green dye, is novel as such and constitutes another aspect of the invention.

Another aspect of the invention comprises a composition for artificially coloring the skin, comprising, in a physiologically acceptable medium, at least a mixture constituted of at least a) a carotene, b) astaxanthin and c) at least one lipophilic green dye.

The methods for artificially coloring the skin can apply to various skin types.

According to this scale, the various skin types that exist can be distinguished according to the following types. They can be classified on the basis of their reactivity to the effects of solar radiation according to the Fitzpatrick scale:

| Type | Skin reactivity | Origin |
|---|---|---|
| I | Always burns, never | Celtic |
| | tans | |
| II | Always burns, tans little | Germanic |
| III | Burns moderately, tans | European |
| | gradually | |
| IV | Burns slightly, tans | Mediterranean |
| | very readily | |
| V | Burns rarely, tans | Middle East - South |
| | intensely | American |
| VI | Never burns, strongly | African |
| | pigmented | |

Method of Coloring with at Least One Ortho-Diphenol:

According to one particular embodiment of the invention, it is possible to apply to the skin:

1) a coloring with a composition (A) based on the mixture of lipophilic dyes which is constituted of at least one carotenoid and of a lipophilic green dye, followed or preceded by;

2) a coloring obtained with a composition (B) comprising, in the physiologically acceptable medium, an effective amount of at least one dye precursor selected from compounds containing at least one aromatic ring having at least two hydroxyl (OH) groups borne by two consecutive carbon atoms of the aromatic ring and an effective amount of a catalytic system comprising a first constituent (B₁) selected from the salts and oxides of Mn(II) and/or Zn(II), and mixtures thereof, and a second constituent (B₂) selected from alkali metal hydrogen carbonates, alkaline-earth metal hydrogen carbonates, and mixtures thereof, the proportions of the first constituent and the second constituent being such that:

$$\frac{[ Mn ( II ) ]}{[ {HCO}_{3} ]} \leq 1 with [ Mn ( II ) ] \neq 0$$

$$\frac{[ Zn ( II ) ]}{[ {HCO}_{3} ]} \leq 1 with [ Zn ( II ) ] \neq 0$$

$$\frac{⌊ Mn ( II ) + Zn ( II ) ⌋}{[ {HCO}_{3} ]} \leq 1 with [ Mn ( II ) ] and [ Zn ( II ) ] \neq 0$$

wherein:

[Mn(II)], [Zn(II)] and [HCO₃] represent, respectively, the molar concentrations of Mn(II), Zn(II) and HCO₃ in the composition.

Generally, the

$$\frac{[ Mn ( II ) ]}{[ {HCO}_{3} ]}$$

ratio ranges from 10⁻⁵ to 10⁻¹, preferably from 10⁻³ to 10⁻², and is typically of the order of 5×10⁻³.

In the case of Zn(II), the

$$\frac{[ Zn ( II ) ]}{[ {HCO}_{3} ]}$$

ratio is, in general, of the order of 10 to 100 times higher than the ratio in the case of Mn(II).

Typically, this ratio is 10⁻⁴ or more, preferably 10⁻³ or more, and preferably of the order of 5×10⁻¹.

In the case of a mixture of Mn(II) and Zn(II), the ratio generally ranges from 10⁻⁵ to 10⁻¹, preferably 10⁻³ to 10⁻², this ratio being selected to be higher when the proportion of Zn(II) in the mixture increases.

Generally, the molar concentration of Mn(II), Zn(II), or Mn(II)+Zn(II) in the final composition ranges from 10⁻³ to 10 mM/l, preferably from 10⁻² to 1 mM/l.

When one or more Mn(II) salts or oxides only is (are) used, the molar concentration of Mn(II) in the final composition is typically from 10⁻³ to 10⁻¹ mM/l, preferably 10⁻² to 10⁻¹ mM/l.

Preferably, when one or more Zn(II) salts or oxides only is (are) used, the concentration of Zn(II) in the final composition is from 5×10⁻² to 10 mM/l, better still from 5×10⁻¹ to 1 mM/l.

Among the Mn(II) and Zn(II) salts suitable for the present invention, exemplary are chlorides, fluorides, iodides, sulfates, phosphates, nitrates and perchlorates, the carboxylic acid salts, and mixtures thereof.

Exemplary are manganese chloride, manganese carbonate (for example, rhodochrosite), Mn(II) difluoride, Mn(II) acetate tetrahydrate, Mn(II) lactate trihydrate, Mn(II) phosphate, Mn(II) iodide, Mn(II) nitrate trihydrate, Mn(II) bromide, Mn(II) perchlorate tetrahydrate and Mn(II) sulfate monohydrate.

The salts that are particularly preferred are MnCl₂ and ZnCl₂.

The carboxylic acid salts also include hydroxylated carboxylic acid salts such as gluconate.

Among the alkali metal hydrogen carbonates and alkaline-earth metal hydrogen carbonates, exemplary are Na hydrogen carbonate, K hydrogen carbonate, Mg hydrogen carbonate and Ca hydrogen carbonate, and mixtures thereof, preferentially Na hydrogen carbonate.

The chemical catalytic system according to this particular mode of coloring obtained with composition (B) constitutes a pseudo-oxydase in that it oxidizes polyphenols, in the presence of oxygen, as would a natural enzyme catalyst having a polyphenoloxydase activity.

The dye precursors of the compositions of the invention are compounds or mixtures of compounds comprising at least one aromatic ring, preferably a benzene ring, containing at least two hydroxyl (OH) groups borne by two consecutive carbon atoms of the aromatic ring.

The aromatic ring may be a condensed aromatic ring optionally containing one or more heteroatoms, such as naphthalene, tetrahydronaphthalene, indane, indene, anthracene, phenanthrene, indol, isoindol, indoline, isoindoline, benzofuran, dihydrobenzofuran, chromane, isochromane, chromene, isochromene, quinoline, tetrahydroquinoline and isoquinoline.

The dye precursors according to the invention can be represented by the formula:

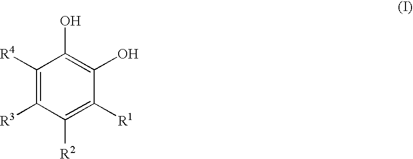

wherein:

the substituents R¹ to R⁴, which may be identical or different, are each a hydrogen atom, a halogen radical, a hydroxyl radical, a carboxyl radical, an alkyl carboxylate radical, an optionally substituted amino radical, an optionally substituted linear or branched alkyl radical, an optionally substituted linear or branched alkenyl radical, an optionally substituted cycloalkyl radical, an alkoxy radical, an alkoxyalkyl radical, an alkoxyaryl radical, it being possible for the aryl group to be optionally substituted, an aryl radical, a substituted aryl radical, an optionally substituted heterocyclic radical, or a radical containing one or more silicon atoms, where two of the substituents R¹ to R⁴ jointly form a saturated or unsaturated ring optionally containing one or more heteroatoms and optionally condensed with one or more saturated or unsaturated rings optionally containing one or more heteroatoms.

The optionally condensed, saturated or unsaturated rings may also be optionally substituted.

The alkyl radicals are generally C₁-C₁₀ alkyl radicals, preferably C₁-C₆ alkyl radicals, such as methyl, ethyl, propyl, butyl, pentyl or hexyl.

The alkoxy radicals are, in general, C₁-C₂₀ alkoxy radicals, such as methoxy, ethoxy, propoxy or butoxy.

The alkoxyalkyl radicals are preferably (C₁-C₂₀)alkoxy(C₁-C₂₀)alkyl radicals, such as methoxymethyl, ethoxymethyl, methoxyethyl, ethoxyethyl, etc.

The cycloalkyl radicals are, in general, C₄-C₈ cycloalkyl radicals, preferably cyclopentyl and cyclohexyl radicals. The cycloalkyl radicals may be substituted cycloalkyl radicals, in particular substituted with alkyl, alkoxy, carboxylic acid, hydroxyl, amine and ketone groups.

The alkenyl radicals are preferably C₁-C₂₀ radicals, such as ethylene, propylene, butylene, pentylene, 2-methyl propylene or decylene.

The radicals containing one or more silicon atoms are preferably polydimethylsiloxane, polydiphenylsiloxane, polydimethylphenylsiloxane or steraoxy dimethicone radicals.

The heterocyclic radicals are, in general, radicals comprising one or more heteroatoms selected from O, N and S, preferably O or N, optionally substituted with one or more alkyl, alkoxy, carboxylic acid, hydroxyl, amine or ketone groups.

Among the preferred heterocyclic radicals, exemplary are furyl, pyranyl, pyrrolyl, imidazolyl, pyrazolyl, pyridyl and thienyl groups.

More preferably, the heterocyclic groups are condensed groups such as benzofuranyl, chromenyl, xanthenyl, indolyl, isoindolyl, quinolyl, isoquinolyl, chromanyl, isochromanyl, indolinyl, isoindolinyl, coumarinyl or isocoumarinyl groups, it being possible for these groups to be substituted, in particular with one or more OH groups.

The preferred dye precursors are:

flavanols, such as catechin and epichatechin gallate,

flavonols, such as quercetin,

anthocyanidins, such as peonidin,

anthocyanins, for example oenin,

hydroxybenzoates, for example gallic acid,

flavones, such as luteolin,

iridoids, such as oleuropein,

it being possible for these products to be osylated (for example glucosylated) and/or in the form of oligomers (procyanidins);

hydroxystilbenes, for example 3,3′,4,5′-tetrahydroxystilbene, which are optionally osylated (for example glucosylated),

3,4-dihydroxyphenylalanine and derivatives thereof,

2,3-dihydroxyphenylalanine and derivatives thereof,

4,5-dihydroxyphenylalanine and derivatives thereof,

4,5-dihydroxyindol and derivatives thereof,

5,6-dihydroxyindol and derivatives thereof,

6,7-dihydroxyindol and derivatives thereof,

2,3-dihydroxyindol and derivatives thereof,

dihydroxycinnamates, such as cafeic acid and chlorogenic acid,

hydroxycoumarins,

hydroxyisocoumarins,

hydroxycoumarones,

hydroxyisocoumarones,

hydroxychalcones,

hydroxychromones,

anthocyans,

quinones,

hydroxyxanthones, and

mixtures thereof.

When the dye precursors have D and L forms, the two forms may be used in the compositions according to the invention.

It is possible to vary the color of the final coloring composition by varying the nature of the various dye precursors and the proportions thereof in the composition. A range of colors is thus obtained.

For example, with a 1/10 ratio of chlorogenic acid and catechin, a light brown coloring is obtained, and with a 1/1 ratio, a mahogany coloring is obtained.

The polymers formed in particular with catechin, gallic acid and their derivatives (tannins) have antimicrobial properties through the tapping of microorganisms during the polymerization. These tannins also have astringent properties that are advantageous for the skin.

The dye precursors may be extracts of plants, fruits, citrus fruits, vegetables and mixtures of these extracts, which contain many phenols as defined above.

Among the plant extracts, exemplary are extracts of rose and of tea.

Among the fruit extracts, exemplary are extracts of apple, of grape (in particular of grapeseed), or cocoa (beans and/or pods) and of banana.

Among the vegetable extracts, exemplary are potato extract.

Use may also be made of mixtures of plant and/or fruit extracts such as mixtures of apple and tea extracts and mixtures of grape and apple extracts.

Depending on the parts of fruits used, for example grapeseeds or pulp, the coloration obtained is different.

The amount of dye precursor in the final composition (D) should be sufficient to obtain a visible coloration. This amount can vary to large extents depending on the nature of the precursor and on the desired intensity for the coloration.

In general, a suitable coloration will be obtained when the amount of dye precursor is such that the content of dye precursor in the final coloring composition is at least 10 micromol per milliliter of final composition.

The physiologically acceptable medium of composition (B) is a solid or liquid medium which is not detrimental to the coloring property of the precursors or to the catalytic effect of the catalytic system.

The physiologically acceptable medium is preferably a medium which solubilizes the dye precursor and which has a bacteriostatic property.

Among the precursor solvents suitable for the formulation of the compositions according to the invention, exemplary are water, alcohols and polar solvents, and mixtures thereof.

The alcohols are preferably lower (C₁-C₆) alkanols, such as ethanol and isopropanol, and alkanediols such as ethylene glycol, propylene glycol and pentanediol.

Among the polar solvents, exemplary are ethers, esters (in particular acetates), dimethyl sulfoxide (DMSO), N-methylpyrrolidone (NMP) and ketones (in particular acetone), and mixtures thereof.

The physiologically acceptable medium of composition (B) preferably comprises water (in particular distilled or deionized water) or a water/alcohol mixture, in particular water/ethanol mixture.

The amount of alcohol in the water/alcohol mixture of composition (B) may represent up to 80% by weight of the water/alcohol mixture, preferably 1% to 50% by weight, and better still 5% to 20% by weight.

The physiologically acceptable medium of composition (B) may be a solid medium such as an excipient for formulating stones and tablets, in particular effervescent stones and tablets.

Preferably, the compositions according to the invention are free of agents for chelating the Mn(II) and/or Zn(II) salts used, since these agents tend to inhibit the oxidation of the dye precursors.

To reveal the coloration of the compositions according to the invention, it is sufficient to place the composition containing at least one dye precursor and an effective amount of the catalytic system according to the invention in contact with an oxidizing medium such as a medium containing oxygen (for example atmospheric oxygen).

According to a first method, a composition (B) comprising all the ingredients of the composition, i.e., both the dye precursor(s) and the catalytic system, is applied to the skin or the keratin fibers, in the presence of oxygen, for example atmospheric oxygen.

According to a second method, a film of a composition B₁ comprising one or more dye precursors as defined above, in a physiologically acceptable medium, may be applied first to the skin or the keratin fibers, and then a film of a composition B₂ comprising the catalytic system in the physiologically acceptable medium, which, in the presence of oxygen, will reveal the coloration of composition B₁, can be applied to the film of base composition.

Obviously, the order of application of the films can be inverted.

The films may be applied by any known means, in particular by spraying.

The compositions (B) may be, and may be conditioned, in various forms.

According to a first embodiment, the compositions (B) may be conditioned in the form of a one-compartment aerosol in which is present the composition containing the dye precursor(s) and the catalytic system, and a standard inert propellant gas such as nitrogen, a saturated hydrocarbon, for instance isopropane, or a fluorohydrocarbon, for example a Freon®.

In a second embodiment, composition (B) may be conditioned in the form of a kit comprising two separate containers, one for the base composition containing the dye precursor(s), the other for the catalytic system, the base composition and the catalytic system being mixed together or applied successively at the time of use.

In a third embodiment, composition (B) may be contained in a one-compartment pump system, without air intake, or in a two-compartment pump system, the base composition being in one compartment and the catalytic system in the other.

In a fourth embodiment, composition (B) may be in the form of stones, in particular bath stones. Each stone may comprise, mixed with an excipient, the dye precursor(s) and the catalytic system, the excipient preventing the reaction in the presence of oxygen or the dye precursor(s) and the catalytic system are contained in different stones.

By breaking down either the single stone or a stone of each of the constituents in water, for example bath water, the coloring composition according to the invention is produced.

As is conventional, the stones may be effervescent stones.

The excipient used may be any standard excipient, such as a mixture of talc, of stearate (in particular magnesium stearate), of citric and/or tartaric acid, and of alkali metal and/or alkaline-earth metal hydrogen carbonate.

The amount of citric and/or tartaric acid present should be such that there is no neutralization of the hydrogen carbonate resulting in a lack of free hydrogen carbonate relative to the Mn(II) and/or Zn(II).

Moreover, as water, in particular tap water and certain spring or mineral waters, generally contain manganese (II), it is sometimes sufficient to place in the water the single stone containing hydrogen carbonate and the dye precursor(s), the Mn(II) content of the catalytic system then being provided by the Mn(II) present in the water.

Similarly, certain plant extracts (for example extracts of tea leaves) may contain large amounts of manganese (II). As a function of these contents, an adjustment of the concentration of the catalytic system is made so as to have a satisfactory result.

Obviously, the intensity of the coloration may be varied by disintegrating several stones in the water.

Furthermore, the rate of coloring may be accelerated by adding to the composition a compound or a formulation of compounds that generate(s) oxygen, for example on contact with water. Thus, such a compound or formulation, for example sodium peroxide, may be incorporated into a stone.

### Galenical Forms:

The compositions of the invention may be in any of the forms suitable for topical application, in particular in the form of aqueous gels, in the form of emulsions obtained by dispersion of a fatty phase (also known as an oily phase) in an aqueous phase (O/W) or conversely (W/O) or multiple emulsions (for example W/O/W or O/W/O or O/O/W). They may be more or less fluid and may have the appearance of a white or colored cream, an ointment, a milk, a lotion, a serum, a paste, a powder or a solid stick, and they may optionally be conditioned as an aerosol and be in the form of a foam or spray. These compositions are prepared according to the usual methods.

According to one particular embodiment of the invention, the compositions of the invention may be in the form of an emulsion and in this case may comprise at least one oily phase. The proportion of the oily phase of the emulsion may range from 1% to 80% by weight, preferably from 2% to 50% by weight, and better still from 2% to 40% by weight, relative to the total weight of the composition. The fatty substances of the oily phase, in particular the oils, and the emulsifiers and coemulsifiers that may be present, used in the composition in emulsion form are selected from those conventionally used in the cosmetics or dermatology field. The emulsifer and the coemulsifier, when they are present, are generally in a proportion ranging from 0.1% to 30% by weight, preferably from 0.3% to 20% by weight, and better still from 0.5% to 15% by weight, relative to the total weight of the composition. The emulsion may also contain lipid vesicules in addition to or instead of the emulsifiers and/or coemulsifiers.

The emulsions generally contain at least one emulsifier selected from amphoteric, anionic, cationic or nonionic emulsifiers, used alone or as a mixture. The emulsifiers are selected in an appropriate manner according to the continuous phase of the emulsion to be obtained (W/O or O/W). When the emulsion is multiple, it generally comprises an emulsifier in the primary emulsion and an emulsifier in the outer phase into which the primary emulsion is introduced.

As emulsifiers that can be used for the preparation of the W/O emulsions, exemplary are, for example, of alkyl esters or ethers of sorbitan, of glycerol or of sugars; silicone surfactants, for instance dimethicone copolyols such as the mixture of cyclomethicone and of dimethicone copolyol marketed under the trademarks DC 5225 C and DC 3225 C by Dow Corning, and for instance alkyl dimethicone copolyols such as the lauryl methicone copolyol marketed under the trademark “Dow Corning 5200 Formulation Aid” by Dow Corning, cetyl dimethicone copolyol marketed under the trademark Abil EM 90® by Goldschmidt, and the mixture of polyglyceryl-4 isostearate/cetyl dimethicone copolyol/hexyl laurate marketed under the trademark Abil WE 09® by Goldschmidt. One or more coemulsifiers may also be added thereto, which coemulsifiers may advantageously be selected from the group comprising branched-chain fatty acid esters of a polyol, and in particular branched-chain fatty acid esters of glycerol and/or of sorbitan, for example polyglyceryl isostearate, such as the product marketed under the trademark Isolan GI 34 by Goldschmidt, sorbitan isostearate, such as the product marketed under the trademark Arlacel 987 by ICI, sorbitan glyceryl isostearate, such as the product marketed under the trademark Arlacel 986 by ICI, and mixtures thereof.

As emulsifiers that may be used for the preparation of the O/W emulsions, exemplary are, for example, of nonionic emulsifiers such as fatty acid esters of oxyalkylenated (more particularly polyoxyethylenated) polyols, and for example polyethylene glycol stearates, for instance PEG-100 stearate, PEG-50 stearate and PEG-40 stearate; oxyalkylenated fatty acid esters of sorbitan comprising, for example, from 20 to 100 EO, for example those marketed under the trademarks Tween 20 or Tween 60 by Uniqema; oxyalkylenated (oxyethylenated and/or oxypropylenated) fatty alcohol ethers; alkoxylated or non-alkoxylated sugar esters, for instance sucrose stearate and such as PEG-20 methylglucose sesquistearate; sorbitan esters such as the sorbitan palmitate marketed under the trademark Span 40 by Uniqema; diacid esters of fatty alcohols, for instance dimyristyl tartrate; mixtures of these emulsifiers, for instance the mixture of glyceryl stearate and of PEG-100 stearate (CTFA name: Glyceryl Stearate/PEG-100 Stearate) marketed under the trademark Arlacel 165 by Uniqema and under the trademark Simulsol 165 by SEPPIC; or the mixture of dimyristyl tartrate, cetearyl alcohol, pareth-7 and PEG-25 laureth-25, marketed under the trademark Cosmacol PSE by Sasol (CTFA name: Dimyristyl tartrate/cetearyl alcohol/12-15 Pareth 7/PPG 25 laureth 25).

Coemulsifiers such as, for example, fatty alcohols having from 8 to 24 carbon atoms, for instance cetyl alcohol, stearyl alcohol and the mixture thereof (cetearyl alcohol), octyldodecanol, 2-butyloctanol, 2-hexyldecanol, 2-undecylpentadecanol or oleyl alcohol, or fatty acids, may be added to these emulsifiers.

It is also possible to prepare emulsions without emulsifying surfactants or containing less than 0.5% thereof relative to the total weight of composition (A) or (B), by using suitable compounds, for stabilizing said emulsions, for example amphiphilic polymers or electrolytes.

### Additives:

When the composition of the invention is in emulsion form, it comprises at least one oily phase which contains at least one oil, in particular a cosmetic oil. The term “oil” means a fatty substance that is liquid at ambient temperature (25° C.).

As oils that can be used in the composition of the invention, it is possible to use, for example, hydrocarbon-based oils of animal origin, such as perhydrosqualene (or squalane); hydrocarbon-based oils of plant origin, such as caprylic/capric acid triglycerides, for instance those marketed by Stearineries Dubois or those marketed under the trademarks Miglyol 810, 812 and 818 by Dynamit Nobel, or, alternatively, oils of plant origin, for instance sunflower oil, maize oil, soybean oil, marrow oil, grapeseed oil, sesame seed oil, hazelnut oil, apricot oil, macadamia oil, arara oil, coriander oil, castor oil, avocado oil, jojoba oil, shey butter oil; synthetic oils; silicone oils, for instance volatile or non-volatile polymethylsiloxanes (PDMs) containing a linear or cyclic silicone chain, which are liquid or pasty at ambient temperature; fluoro oils, such as partially hydrocarbon-based and/or silicone-based fluoro oils, for instance those described in JP-A-2-295912; ethers, such as dicapryl ether (CTFA name: Dicaprylyl ether); C₁₂-C₁₅ fatty alkyl benzoates (Finsolv TN from Finetex); arylalkyl benzoate derivatives, such as 2-phenylethyl benzoate (X-Tend 226 from ISP); amido oils, for instance isopropyl N-lauroylsarcosinate (Eldew SL-205 from Ajimoto); and mixtures thereof.

The compositions of the invention may also contain one or more organic solvents that may be selected from the group constituted of hydrophilic organic solvents, lipophilic organic solvents and amphiphilic organic solvents, or mixtures thereof.

Among the hydrophilic organic solvents, mention may, for example, be made of linear or branched monohydric alcohols having from 1 to 8 carbon atoms, for instance ethanol, propanol, butanol, isopropanol or isobutanol; polyethylene glycols having from 6 to 80 ethylene oxides; polyols such as propylene glycol, isoprene glycol, butylene glycol, glycerol or sorbitol; monoalkyl or dialkyl isosorbides in which the alkyl groups contain from 1 to 5 carbon atoms, for instance dimethyl isosorbide; glycol ethers, for instance diethylene glycol monomethyl ether or monoethyl ether and propylene glycol ethers, for instance dipropylene glycol methyl ether.

Amphiphilic organic solvents that are exemplary include polypropylene glycol (PPG) derivatives such as fatty acid esters of polypropylene glycol, and derivatives of PPG and of fatty alcohols, for instance PPG-23 oleyl ether, and PPG-36 oleate.

Examples of lipophilic organic solvents include fatty esters such as diisopropyl adipate, dioctyl adipate or alkyl benzoates.

The compositions in accordance with the present invention may also comprise conventional cosmetic adjuvants selected from demulcents, humectants, opacifiers, stabilizers, emollients, silicones, antifoaming agents, fragrances, preservatives, anionic, cationic, nonionic, zwitterionic or amphoteric surfactants, fillers, polymers, propellants, acidifying or basifying agents, or any other ingredient normally used in the cosmetics and/or dermatological field.

Hydrophilic thickeners that are exemplary include carboxyvinyl polymers such as carbopols (carbomers), the Pemulen products (acrylate/C10-C30-alkylacrylate copolymer) and homopolymers and copolymers of acrylamide and/or of 2-acrylamido-2-methylpropanesulfonic acid (AMPS), for instance sodium polyacryloyldimethyltaurate (and) polysorbate 80 (and) sorbitan oleate marketed under the trademark Simulgel 800 by SEPPIC; cellulose derivatives such as hydroxyethylcellulose; polysaccharides and in particular gums such as xanthan gum; and mixtures thereof.

Lipophilic thickeners that are exemplary include modified clays such as hectorite and derivatives thereof, for instance the products marketed under the trademark Bentone.

Preservatives that are exemplary include para-hydroxybenzoic acid esters, also known as Parabens® (in particular methyl paraben, ethyl paraben and propyl paraben), phenoxyethanol, formaldehyde generators, for instance imidazolidinylurea or diazolidinylurea, chlorhexidine digluconate, sodium benzoate, caprylyl glycol, iodo propynyl butyl carbamate, pentylene glycol, alkyltrimethylammonium bromides such as myristyltrimethylammonium bromide (CTFA name: myrtrimonium bromide), dodecyltrimethylammonium bromide, hexadecyltrimethylammonium bromide, and mixtures thereof, such as the mixture marketed under the trademark Cetrimide® by FEF Chemicals. The preservative may be present in the composition according to the invention in a content ranging from 0.001% to 10% by weight, relative to the total weight of the composition, especially ranging from 0.1% to 5% by weight, and in particular ranging from 0.2% to 3% by weight.

According to one particular embodiment of the invention, to improve the stability of dehydroascorbic acid or a polymer thereof and/or that of ascorbic acid or a salt or derivative thereof, each of these active agents may be encapsulated according to standard encapsulation techniques.

### Self-Tanning Agents:

According to one particular embodiment of the invention, the compositions containing the mixture of lipophilic dyes which is constituted of at least one carotenoid and of a lipophilic green dye may also comprise one or more additional self-tanning agents.

The self-tanning agents are generally selected from monocarbonyl or polycarbonyl compounds such as, for example, isatin, alloxan, ninhydrin, glyceraldehyde, mesotartaric aldehyde, glutaraldehyde, erythrulose, the pyrazolin-4,5-dione derivatives as described in FR-2,466,492 and WO 97/35842, dihydroxyacetone (DHA), and the 4,4-dihydroxypyrazolin-5-one derivatives as described in EP-903,342. DHA will preferably be used.

The DHA may be used in free form and/or in a form encapsulated, for example, in lipid vesicules such as liposomes, in particular described in WO 97/25970.

The self-tanning agent(s) is (are) generally present in proportions ranging from 0.1% to 15% by weight, and preferably from 0.2% to 10% by weight, and more preferentially from 1% to 8% by weight, relative to the total weight of the composition.

### Stabilizers:

To improve the stability of the compositions in accordance with the invention, said compositions may also include one or more stabilizers.

Examples of stabilizers include:

(1) antioxidants,

(2) chelating agents,

(3) non-crosslinked N-vinylimidazole polymers or copolymers such as those described in EP-1,316,302.

According to the invention, the expression “non-crosslinked N-vinylimidazole polymer or copolymer” means any polymer comprising N-vinylimidazole units, and not comprising a crosslinking agent. Copolymers that are suitable for use in the invention are, for example, copolymers comprising N-vinylimidazole units and N-vinylpyrrolidone and/or N-vinylcaprolactam units.

In one advantageous aspect of the invention, the copolymer has a mole fraction of N-vinylimidazole units of from 0.1 and 1 and more preferentially from 0.4 to 0.9.

According to one advantageous aspect of the invention, the mole ratio from the N-vinylimidazole unit equivalent and the oxidation-sensitive hydrophilic active agent ranges from 0.004 to 16 and preferentially from 0.01 to 1.

An N-vinylimidazole/N-vinylpyrrolidone copolymer will preferentially be used.

The weight-average molar mass of the N-vinylimidazole polymers will advantageously be from 1000 and 1×10⁷ and preferably from 5000 and 5×10⁶.

The vinylpyrrolidone/vinylimidazole (50/50) copolymer with a weight-average molar mass of 1,200,000 marketed under the reference Luvitec VPI 55K72W by BASF or the vinylpyrrolidone/vinylimidazole (50/50) copolymer with a weight-average molar mass of 10,000 marketed under the reference Luvitec VPI 55K18P by BASF may be used for this purpose. The polymers or copolymers according to the invention may be prepared, for example, according to the method described in WO 97/45517.

(4) Amphiphilic polymers selected from polyisobutylene-based oligomers or polymers comprising a polyisobutylene apolar portion containing at least 40 carbon atoms and at least one polar end portion constituted of carboxylic or dicarboxylic acids, anhydrides thereof or modified forms thereof in the form of esters, amides or salts, and mixtures thereof as described in EP-1,481,677, can also be used as stabilizers.

These amphiphilic polymers are constituted of a polyisobutylene apolar portion and of at least one polar portion.

The polyisobutylene apolar portion contains at least 40 carbon atoms and preferably from 60 to 700 carbon atoms. It is important for this portion to contain at least 40 carbon atoms to achieve the aim of the invention. If there are less than 40 carbon atoms, a satisfactorily stable system is not obtained.

The polar portion of these amphiphilic polymers or oligomers is constituted of carboxylic or dicarboxylic acids, anhydrides thereof or modified forms thereof in the form of esters, amides or salts, and mixtures thereof. Preferably, the polar end portion is constituted of dicarboxylic acids or anhydrides thereof or of modified forms thereof in the form of esters, amides or salts.

The expression “modified forms in the form of esters, amides or salts” is carboxylic or dicarboxylic acids modified with alcohols, amines, alkanolamines or polyols, or, alternatively, in the form of alkali metal, alkaline-earth metal or ammonium salts or, alternatively, in the form of salts of an organic base, for instance the diethanolamine and triethanolamine salts.

The oligomers or polymers derived from succinic acid or anhydride may be selected especially from the polyisobutylene derivatives of succinic acid or anhydride described in U.S. Pat. Nos. 4,234,435, 4,708,753, 5,129,972, 4,931,110, GB-A-2,156,799 and 4,919,179. The polyisobutylene portion may be hydrogenated or non-hydrogenated, with a molecular weight ranging from 400 to 5000. In the succinic-terminated polyisobutylene thus obtained, the succinic portion may be esterified, amidated or in salt form, i.e., it may be advantageously modified with alcohols, amines, alkanolamines or polyols, or, alternatively, may be in the form of alkali metal, alkaline-earth metal or ammonium salts or, alternatively, in the form of salts of an organic base, for instance the diethanolamine and triethanolamine salts. The esterified or amidated succinic-terminated polyisobutylenes are products of reaction of (a) a polyisobutylene containing succinic end groups, and (b) an amine or an alcohol, to form an amide or an ester. The term “amine” used herein includes all types of amines, including alkanolamines. They may be, for example, primary, secondary or tertiary monoamines, these amines possibly being aliphatic, cycloaliphatic, aromatic or heterocyclic, and saturated or unsaturated. Moreover, the alcohols may be monoalcohols or polyalcohols. The monoalcohols comprise primary, secondary or tertiary aliphatic alcohols, and phenols. The polyalcohols may be selected, for example, from aliphatic, cycloaliphatic, aromatic and heterocyclic polyalcohols. The modified (esterified or amidated) succinic-terminated polyisobutylenes and the process for preparing them are described in particular in U.S. Pat. No. 4,708,753.

Succinic-terminated polyisobutylenes that may especially be mentioned include modified succinic-terminated polyisobutylenes, such as the products marketed under the trademarks Lubrizol 5603 and Lubrizol 2650 by Lubrizol. According to one preferred embodiment of the invention, the polymer marketed under the trademark Lubrizol 5603 by Lubrizol, which is the diethylethanolamine salt of esterified succinic-terminated polyisobutylene (INCI name: Hydroxyethyldiethonium polyisobutenyl triethylaminosuccinate/diethylethanolamine), is used.

Another example of a polyisobutylene derivative that may be used in the invention is the product of reaction of maleic anhydride with polyisobutylene, such as the product marketed under the trademark Glissopal SA by BASF.

(5) Maleic anhydride copolymers comprising one or more maleic anhydride comonomers and one or more comonomers selected from vinyl acetate, vinyl alcohol, vinylpyrrolidone, olefins having from 2 to 20 carbon atoms and styrene, as described in EP-1,374,849, may also be mentioned as stabilizers.

According to the invention, the term “maleic anhydride copolymer” means any polymer obtained by copolymerization of one or more maleic anhydride comonomers and of one or more comonomers selected from vinyl acetate, vinyl alcohol, vinylpyrrolidone, olefins having from 2 to 20 carbon atoms, for instance octadecene, ethylene, isobutylene, diisobutylene, isooctylene, and styrene, the maleic anhydride comonomers being optionally partially or totally hydrolysed. Preferably, hydrophilic polymers will be used, i.e., polymers with a solubility in water of greater than or equal to 2 g/l.

Copolymers that are more particularly suitable for use in the invention are copolymers obtained by copolymerization of one or more maleic anhydride units, whose maleic anhydride units are in hydrolyzed form, and preferentially in the form of alkaline salts, for example in the form of ammonium, sodium, potassium or lithium salts.

In one advantageous aspect of the invention, the copolymer has a mole fraction of maleic anhydride units of from 0.1 to 1 and more preferentially from 0.4 to 0.9.

According to one advantageous aspect of the invention, the mole ratio from the maleic anhydride unit equivalent and the oxidation-sensitive hydrophilic active agent ranges from 0.005 to 10 and preferentially from 0.01 to 1

The weight-average molar mass of the maleic anhydride copolymers will advantageously be from 1,000 to 500,000 and preferably from 1,000 to 50,000.

Preferentially, a copolymer of styrene and of maleic anhydride in a 50/50 ratio will be used.

The styrene/maleic anhydride (50/50) copolymer, in the form of the ammonium salt at 30% in water, marketed under the reference SMA1000H® by Atofina, or the styrene/maleic anhydride (50/50) copolymer, in the form of the sodium salt at 40% in water, marketed under the reference SMA1000HNa® by Atofina, may be used, for example.

### Photoprotective Agents:

The compositions in accordance with the invention may also include one or more photoprotective agents.

The photoprotective agents in accordance with the invention are selected from UVA-active and/or UVB-active organic and/or inorganic UV-screening agents that are hydrophilic and/or lipophilic and/or insoluble in the cosmetic solvents commonly used.

The organic UV-screening agents are in particular selected from cinnamic derivatives; anthranilates; salicylic derivatives, dibenzoylmethane derivatives, camphor derivatives; benzophenone derivatives; β,β-diphenylacrylate derivatives; triazine derivatives; benzotriazole derivatives; benzalmalonate derivatives, in particular those cited in U.S. Pat. No. 5,624,663; benzimidazole derivatives; imidazolines; bisbenzoazolyl derivatives as described in EP-669,323 and U.S. Pat. No. 2,463,264; p-aminobenzoic acid (PABA) derivatives; methylene bis(hydroxyphenylbenzotriazole) derivatives as described in U.S. Pat. Nos. 5,237,071, 5,166,355, GB-2,303,549, DE-197,26,184 and EP-893,119; benzoxazole derivatives as described in EP-0,832,642, EP-1,027,883, EP-1,300,137 and DE-101,62,844; screening polymers and screening silicones such as those described in particular in WO 93/04665; α-alkylstyrene-derived dimers, such as those described in DE-198,55,649; 4,4-diarylbutadienes such as those described in applications EP-0,967,200, DE-197,46,654, DE-197,55,649, EP-A-1,008,586, EP-1,133,980 and EP-133,981; merocyanin derivatives such as those described in WO 04/006878, WO 05/058269 and WO 06/032741, and mixtures thereof.

As examples of additional organic photoprotective agents, exemplary are those denoted hereinbelow under their INCI name:

### Cinnamic Derivatives:

Ethylhexyl methoxycinnamate marketed in particular under the trademark Parsol MCX by Hoffmann Laroche,

Isopropyl methoxycinnamate,

Isoamyl methoxycinnamate marketed under the trademark Neo Heliopan E 1000 by Haarmann and Reimer,

DEA methoxycinnamate,

Diisopropyl methylcinnamate,

Glyceryl ethylhexanoate dimethoxycinnamate.

### Dibenzoylmethane Derivatives:

Butylmethoxydibenzoylmethane marketed in particular under the trademark Parsol 1789 by Hoffmann Laroche,

Isopropyldibenzoylmethane.

Para-aminobenzoic acid derivatives:

PABA,

Ethyl PABA,

Ethyl dihydroxypropyl PABA,

Ethylhexyl dimethyl PABA marketed in particular under the trademark “Escalol 507” by ISP,

Glyceryl PABA,

PEG-25 PABA marketed under the trademark “Uvinul P25” by BASF.

### Salicylic Derivatives:

Homosalate marketed under the trademark “Eusolex HMS” by Rona/EM Industries,

Ethylhexyl salicylate marketed under the trademark “Neo Heliopan OS” by Haarmann and Reimer,

Dipropylene glycol salicylate marketed under the trademark “Dipsal” by Scher,

TEA salicylate, marketed under the trademark “Neo Heliopan TS” by Haarmann and Reimer.

β,β-diphenylacrylate Derivatives:

Octocrylene marketed in particular under the trademark “Uvinul N539” by BASF,

Etocrylene, marketed in particular under the trademark “Uvinul N35” by BASF.

### Benzophenone Derivatives:

Benzophenone-1 marketed under the trademark “Uvinul 400” by BASF,

Benzophenone-2 marketed under the trademark “Uvinul D50” by BASF,

Benzophenone-3 or oxybenzone, marketed under the trademark “Uvinul M40” by BASF,

Benzophenone-4 marketed under the trademark “Uvinul MS40” by BASF,

Benzophenone-5,

Benzophenone-6 marketed under the trademark “Helisorb 11” by Norquay,

Benzophenone-8 marketed under the trademark “Spectra-Sorb UV-24” by American Cyanamid,

Benzophenone-9 marketed under the trademark “Uvinul DS-49” by BASF,

Benzophenone-12,

n-hexyl 2-(4-diethylamino-2-hydroxybenzoyl)benzoate marketed under the trademark “Uvinul A+” or as a mixture with octylmethoxycinnamate under the trademark “Uvinul A+B” by BASF.

### Benzylidenecamphor Derivatives:

3-Benzylidenecamphor manufactured under the trademark “Mexoryl SD” by Chimex,

4-Methylbenzylidenecamphor marketed under the trademark “Eusolex 6300” by Merck,

Benzylidenecamphorsulfonic acid manufactured under the trademark “Mexoryl SL” by Chimex,

Camphor benzalkonium methosulfate manufactured under the trademark “Mexoryl SO” by Chimex,

Terephthalylidenedicamphorsulfonic acid manufactured under the trademark “Mexoryl SX” by Chimex,

Polyacrylamidomethylbenzylidenecamphor manufactured under the trademark “Mexoryl SW” by Chimex.

### Phenylbenzimidazole Derivatives:

Phenylbenzimidazolesulfonic acid marketed in particular under the trademark “Eusolex 232” by Merck,

Disodium phenyl dibenzimidazole tetrasulfonate marketed under the trademark “Neo Heliopan AP” by Haarmann and Reimer.

### Phenylbenzotriazole Derivatives:

Drometrizole trisiloxane marketed under the trademark “Silatrazole” by Rhodia Chimie,

Methylenebis(benzotriazolyl)tetramethylbutylphenol marketed in solid form under the trademark “MIXXIM BB/100” by Fairmount Chemical, or in micronized form as an aqueous dispersion under the trademark “Tinosorb M” by Ciba Speciality Chemicals.

### Triazine Derivatives:

bis-Ethylhexyloxyphenol methoxyphenyl triazine marketed under the trademark “Tinosorb S” by Ciba Geigy,
ethylhexyl triazone marketed in particular under the trademark “Uvinul T150” by BASF,
diethylhexyl butamido triazone marketed under the trademark “Uvasorb Heb” by Sigma 3V,
2,4,6-tris(dineopentyl 4′-aminobenzalmalonate)-s-triazine,
2,4,6-tris(diisobutyl 4′-aminobenzalmalonate)-s-triazine,
2,4-bis(n-butyl 4′-aminobenzoate)-6-(aminopropyltrisiloxane)-s-triazine,
2,4-bis(dineopentyl 4′-aminobenzalmalonate)-6-(n-butyl 4′-aminobenzoate)-s-triazine,
2,4-bis(n-butyl 4′-diylaminobenzoate)-6{[1,3,3,3-tetramethyl-1-[(trimethylsilyl)oxy]disiloxanyl]propyl-3-ylamino}-2-triazine,
the symmetrical triazine screening agents described in U.S. Pat. No. 6,225,467, WO 2004/085412 (see compounds 6 and 9) or the document “Symmetrical Triazine Derivatives” IP.COM Journal, IP.COM INC West Henrietta, N.Y., US (20 Sep. 2004), especially 2,4,6-tris(biphenyl)-1,3,5-triazines, in particular 2,4,6-tris(biphenyl-4-yl-1,3,5-triazine) and 2,4,6-tris(terphenyl)-1,3,5-triazine which is also mentioned in WO 06/035000, WO 06/034982, WO 06/034991, WO 06/035007, WO 2006/034992 and WO 2006/034985.

### Anthranilic Derivatives:

Menthyl anthranilate marketed under the trademark “Neo Heliopan MA” by Haarmann and Reimer.

### Imidazoline Derivatives:

Ethylhexyldimethoxybenzylidenedioxoimidazoline propionate.

### Benzalmalonate Derivatives:

Polyorganosiloxane containing benzalmalonate functions, for instance the Polysilicone-15 marketed under the trademark “Parsol SLX” by Hoffmann La Roche.

4,4-diarylbutadiene derivatives:
1,1-dicarboxy(2,2′-dimethylpropyl)-4,4-diphenylbutadiene.

### Benzoxazole Derivatives:

2,4-bis[5-(1-dimethylpropyl)benzoxazol-2-yl(4-phenyl)imino]-6-(2-ethylhexyl)imino-1,3,5-triazine marketed under the trademark Uvasorb K2A by Sigma 3V.

### Merocyanin Derivatives:

Octyl-5-N,N-diethylamino-2-phenylsulfonyl-2,4-pentadienoate, and mixtures thereof.
The preferential organic UV-screening agents are selected from:
Ethylhexyl methoxycinnamate,
Ethylhexyl salicylate,
Homosalate,
Butylmethoxydibenzoylmethane,
Octocrylene,
Phenylbenzimidazolesulfonic acid,
Benzophenone-3,
Benzophenone-4,
Benzophenone-5,
n-Hexyl 2-(4-diethylamino-2-hydroxybenzoyl)benzoate,
4-Methylbenzylidenecamphor,
Terephthalylidenedicamphorsulfonic acid,
Disodium phenyl dibenzimidazole tetrasulfonate,
Methylenebis(benzotriazolyl)tetramethylbutylphenol,
bis-Ethylhexyloxyphenol methoxyphenol triazine,
Ethylhexyl triazone,
Diethylhexyl butamido triazone,
2,4,6-tris(dineopentyl 4′-aminobenzalmalonate)-s-triazine,
2,4,6-tris(diisobutyl 4′-aminobenzalmalonate)-s-triazine,
2,4-bis(n-butyl 4′-aminobenzoate)-6-(aminopropyltrisiloxane)-s-triazine,
2,4-bis(dineopentyl 4′-aminobenzalmalonate)-6-(n-butyl 4′-aminobenzoate)-s-triazine,
2,4-bis(n-butyl 4′-diylaminobenzoate)-6-{[1,3,3,3-tetramethyl-1-[(trimethylsilyl)oxy]disiloxanyl]propyl-3-ylamino}-s-triazine,
2,4,6-tris(biphenyl-4-yl-1,3,5-triazine),
2,4,6-tris(terphenyl)-1,3,5-triazine,
Drometrizole trisiloxane,
Polysilicone-15,
1,1-Dicarboxy(2,2′-dimethylpropyl)-4,4-diphenylbutadiene,
Octyl-5-N,N-diethylamino-2-phenylsulfonyl-2,4-pentadienoate,
2,4-bis[5-(1-dimethylpropyl)benzoxazol-2-yl(4-phenyl)imino]-6-(2-ethylhexyl)imino-1,3,5-triazine,
and mixtures thereof.

The inorganic UV-screening agents used in accordance with the present invention are coated or uncoated metal oxide pigments, for instance pigments of titanium oxide (amorphous or crystallized in rutile and/or anatase form), of iron oxide, of zinc oxide, of zirconium oxide or of cerium oxide. Preferably, the inorganic UV-screening agents of the invention are particles of metal oxide having a mean elementary particle size of less than or equal to 500 nm, more preferentially from 5 nm to 500 nm, and even more preferentially from 10 nm to 100 nm, and even more particularly from 15 to 50 nm.

The pigments may be coated or uncoated.

The coated pigments are pigments that have undergone one or more surface treatments of chemical, electronic, mechanochemical and/or mechanical nature with compounds as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal alkoxides (of titanium or of aluminum), polyethylene, silicones, proteins (collagen, elastin), alkanolamines, silicon oxides, metal oxides or sodium hexametaphosphate.

As is known, silicones are organosilicon polymers or oligomers of linear or cyclic, branched or crosslinked structure, of variable molecular weight, obtained by polymerization and/or polycondensation of suitably functionalized silanes, and consist essentially of a repetition of main units in which the silicon atoms are linked together via oxygen atoms (siloxane bond), optionally substituted hydrocarbon-based radicals being directly attached via a carbon atom to said silicon atoms.

The term “silicones” also includes the silanes required for their preparation, in particular alkyl silanes.

The silicones used for coating the pigments that are suitable for the present invention are preferably selected from the group containing alkyl silanes, polydialkylsiloxanes and polyalkylhydrogenosiloxanes. Even more preferentially, the silicones are selected from the group containing octyltrimethylsilane, polydimethylsiloxanes and polymethylhydrogenosiloxanes.

Needless to say, before being treated with silicones, the metal oxide pigments may have been treated with other surface agents, in particular with cerium oxide, alumina, silica, aluminum compounds or silicon compounds, or mixtures thereof.

The coated pigments are more particularly titanium oxides that have been coated:

with silica, such as the product Sunveil from the company Ikeda,

with silica and iron oxide, such as the product Sunveil F from the company Ikeda,

with silica and alumina, such as the products Microtitanium Dioxide MT 500 SA and Microtitanium Dioxide MT 100 SA from the company Tayca, Tioveil from the company Tioxide and Mirasun TiW 60 from the company Rhodia,

with alumina, such as the products Tipaque TTO-55 (B) and Tipaque TTO-55 (A) from the company Ishihara and UVT 14/4 from the company Kemira,

with alumina and aluminum stearate, such as the product Microtitanium Dioxide MT 100 T, MT 100 TX, MT 100 Z and MT-01 from the company Tayca, the products Solaveil CT-10 W and Solaveil CT 100 from the company Uniqema, and the product Eusolex T-AVO from the company Merck,

with silica, alumina and alginic acid, such as the product MT-100 AQ from the company Tayca,

with alumina and aluminum laurate, such as the product Microtitanium Dioxide MT 100 S from the company Tayca,

with iron oxide and iron stearate, such as the product Microtitanium Dioxide MT 100 F from the company Tayca,

with zinc oxide and zinc stearate, such as the product BR351 from the company Tayca,

with silica and alumina and treated with a silicone, such as the products Microtitanium Dioxide MT 600 SAS, Microtitanium Dioxide MT 500 SAS or Microtitanium Dioxide MT 100 SAS from the company Tayca,

with silica, alumina and aluminum stearate and treated with a silicone, such as the product STT-30-DS from the company Titan Kogyo,

with silica and treated with a silicone, such as the product UV-Titan X 195 from the company Kemira,

with alumina and treated with a silicone, such as the products Tipaque TTO-55 (S) from the company Ishihara or UV Titan M 262 from the company Kemira,

with triethanolamine, such as the product STT-65-S from the company Titan Kogyo,

with stearic acid, such as the product Tipaque TTO-55 (C) from the company Ishihara,

with sodium hexametaphosphate, such as the product Microtitanium Dioxide MT 150 W from the company Tayca.

Other titanium oxide pigments treated with a silicone are preferably TiO₂ treated with octyltrimethylsilane and for which the mean size of the elementary particles is from 25 and 40 nm, such as the product marketed under the trademark T 805 by Degussa Silices, TiO₂ treated with a polydimethylsiloxane and for which the mean size of the elementary particles is 21 nm, such as the product marketed under the trademark 70250 Cardre UF TiO₂SI₃ by Cardre, anatase/rutile TiO₂ treated with a polydimethylhydrogenosiloxane and for which the mean size of the elementary particles is 25 nm, such as the product marketed under the trademark Microtitanium Dioxide USP Grade Hydrophobic by Color Techniques.

The uncoated titanium oxide pigments are marketed, for example, by Tayca under the trademarks Microtitanium Dioxide MT 500 B or Microtitanium Dioxide MT 600 B, by Degussa under the trademark P 25, by Wacker under the trademark Transparent titanium oxide PW, by Miyoshi Kasei under the trademark UFTR, by Tomen under the trademark ITS and by Tioxide under the trademark Tioveil AQ.

The uncoated zinc oxide pigments are, for example:

those marketed under the trademark Z-Cote by Sunsmart;

those marketed under the trademark Nanox by Elementis;

those marketed under the trademark Nanogard WCD 2025 by Nanophase Technologies.

The coated zinc oxide pigments are, for example:

those marketed under the trademark Zinc Oxide CS-5 by Toshibi (ZnO coated with polymethylhydrogenosiloxane);

those marketed under the trademark Nanogard Zinc Oxide FN by Nanophase Technologies (as a 40% dispersion in Finsolv TN, C₁₂-C₁₅ alkyl benzoate);

those marketed under the trademark Daitopersion ZN-30 and Daitopersion ZN-50 by Daito (dispersions in cyclopolymethylsiloxane/oxyethylenated polydimethylsiloxane, containing 30% or 50% of nanozinc oxides coated with silica and polymethylhydrogenosiloxane);

those marketed under the trademark NFD Ultrafine ZNO by Daikin (ZnO coated with perfluoroalkyl phosphate and copolymer based on perfluoroalkylethyl as a dispersion in cyclopentasiloxane);

those marketed under the trademark SPD-Z1 by Shin-Etsu (ZnO coated with silicone-grafted acrylic polymer, dispersed in cyclodimethylsiloxane);

those marketed under the trademark Escalol Z100 by ISP (alumina-treated ZnO dispersed in an ethylhexyl methoxycinnamate/PVP-hexadecene/methicone copolymer mixture);

those marketed under the trademark Fuji ZnO-SMS-10 by Fuji Pigment (ZnO coated with silica and polymethylsilsesquioxane);

those marketed under the trademark Nanox Gel TN by Elementis (ZnO dispersed at a concentration of 55% in C₁₂-C₁₅ alkyl benzoate with hydroxystearic acid polycondensate).

The uncoated cerium oxide pigments are marketed, for example, under the trademark Colloidal Cerium Oxide by Rhone-Poulenc. The uncoated iron oxide nanopigments are marketed, for example, by Arnaud under the trademarks Nanogard WCD 2002 (FE 45B), Nanogard Iron FE 45 BL AQ, Nanogard FE 45R AQ and Nanogard WCD 2006 (FE 45R) or by Mitsubishi under the trademark TY-220.

The coated iron oxide pigments are marketed, for example, by Arnaud under the trademarks Nanogard WCD 2008 (FE 45B FN), Nanogard WCD 2009 (FE 45B 556), Nanogard FE 45 BL 345 and Nanogard FE 45 BL or by BASF under the trademark Transparent Iron Oxide.

Mention may also be made of mixtures of metal oxides, especially of titanium dioxide and of cerium dioxide, including the silica-coated equal-weight mixture of titanium dioxide and of cerium dioxide, marketed by Ikeda under the trademark Sunveil A, and also the alumina, silica and silicone-coated mixture of titanium dioxide and of zinc dioxide, such as the product M 261 marketed by Kemira, or the alumina, silica and glycerol-coated mixture of titanium dioxide and of zinc dioxide, such as the product M 211 marketed by Kemira.

The photoprotective agents are generally present in the compositions containing dehydroascorbic acid or a polymer thereof and/or the compositions comprising ascorbic acid or a salt or derivative thereof in proportions ranging from 0.01% to 20% by weight relative to the total weight of the composition, and preferably ranging from 0.1% to 10% by weight relative to the total weight of the composition.

### Additional Coloring Agents:

To nuance the color obtained, the compositions of the invention may also comprise one or more additional coloring agents.

The additional coloring agents may also be selected from natural or synthetic direct dyes. They may be organic or inorganic dyes.

The natural or synthetic liposoluble organic dyes are, for example, DC Red 17, DC Red 21, DC Red 27, DC Yellow 11, DC Violet 2, DC Orange 5, Soudan red, palm oil, Soudan brown, quinoline yellow, annatto and curcumin.

The natural or synthetic water-soluble dyes are, for example, FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanin (beetroot), carmine, copper-containing chlorophylline, methylene blue, anthocyanins (enocyanin, black carrot, hibiscus or elder) and riboflavin.

The dyes may also be selected from anthraquinones, caramel, carmine, carbon black, azulene blues, methoxalenez, trioxalene, guajazulene, chamuzulene, rose Bengal, cosine 10B, cyanosin, daphinine, juglone, lawsone, extracts of fermented soya, of algae, of fungi or of microorganisms, flavylium salts not substituted in position 3, for instance those described in EP-1,172,091, extracts of Gesneria fulgens, Blechum procerum or Saxifraga and pigments that may be obtained by extraction with an organic or aqueous-organic solvent of a culture medium of micromycetes of the Monascus type.

These dyes may also be selected from indole derivatives, for instance the monohydroxyindoles as described in FR-2,651,126 (i.e., 4-, 5-, 6- or 7-hydroxyindole) or the dihydroxyindoles as described in EP-B-0,425,324 (i.e., 5,6-dihydroxyindole, 2-methyl-5,6-dihydroxyindole, 3-methyl-5,6-dihydroxyindole or 2,3-dimethyl-5,6-dihydroxyindole).

These dyes may also be dyes obtained with compounds comprising at least one aromatic ring containing at least two hydroxyl groups (OH) borne by two consecutive carbon atoms of the aromatic ring and a catalytic system comprising a first constituent selected from Mn(II) and/or Zn(II) salts and oxides, and mixtures thereof, and a second constituent selected from alkali metal hydrogen carbonates, alkaline-earth metal hydrogen carbonates, and mixtures thereof, as described previously.

The additional coloring agents may also be selected from particulate dyestuffs, which are preferably selected from pigments, pearlescent agents or interference pigments, and glitter flakes.

The term “pigments” should be understood as meaning white or colored, mineral or organic particles of any form, which are insoluble in the physiological medium and are intended to color the composition.

The pigments may be white or colored, and mineral and/or organic. Among the mineral pigments that are exemplary are titanium dioxide, optionally surface-treated, zirconium oxide or cerium oxide, and also zinc oxide, iron (black, yellow or red) oxide or chromium oxide, manganese violet, ultramarine blue, chromium hydrate and ferric blue, and metal powders, for instance aluminum powder and copper powder.

Among the organic pigments that are exemplary are carbon black, pigments of D&C type and lakes based on cochineal carmine or on barium, strontium, calcium or aluminum.

Mention may also be made of pigments with an effect, such as particles comprising a natural or synthetic, organic or mineral substrate, for example glass, acrylic resins, polyester, polyurethane, polyethylene terephthalate, ceramics or aluminas, said substrate optionally being coated with metallic substances such as aluminum, gold, silver, platinum, copper or bronze, or metal oxides such as titanium dioxide, iron oxide or chromium oxide, and mixtures thereof.

For the purposes of the present invention, the term “interference particles or pearlescent agents” is any particle generally having a multilayer structure such that it allows the creation of a color effect by interference of light rays, which diffract and scatter differently according to the nature of the layers. The coloring effects obtained are associated with the lamellar structure of these particles and are derived from the physical laws of thin film optics (see: Pearl Lustre Pigments—Physical principles, properties, applications—R. Maisch, M. Weigand. Verlag Moderne Industrie). Thus, these particles may have colors that vary according to the angle of observation and the incidence of the light.

For the purposes of the present invention, a multilayer structure is intended to denote, without preference, a structure formed from a substrate coated with a single layer, or a structure formed from a substrate coated with at least two or even more consecutive layers.

The multilayer structure may thus comprise one or even at least two layers, each layer, independently or otherwise of the other layer(s), being made of at least one material selected from the group constituted of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys and polymers, and combinations thereof.

Generally, the multilayer structure is of mineral nature.

More particularly, the interference particles under consideration according to the invention may be interference pigments, or, alternatively, natural or synthetic, monolayer or multilayer pearlescent agents, in particular formed from a natural substrate based, inter alia, on mica, which is covered with one or more layers of metal oxide.

The interference particles according to the invention are characterized in that 50% of the mass population has a diameter (d50) of less than 40 μm, more particularly less than 30 μm, especially less than 20 μm and in particular less than 15 μm, measured with a laser granulometer, for instance the Mastersizer 2000® machine from Malvern or the BI90+® machine from Brookhaven Instruments Corporation.

Pearlescent agents of mica/tin oxide/titanium oxide type, for instance those marketed under the trademarks Timiron Silk Blue®, Timiron Silk Red®, Timiron Silk Green®, Timiron Silk Gold® and Timiron Super Silk® marketed by Merck, and mica/iron oxide/titanium oxide pearlescent agents, for instance Flamenco Satin Blue®, Flamenco Satin Red® and Flamenco Satin Violet® and Flamenco Orange 320C marketed by Engelhard, and mixtures thereof, are most particularly suitable for the invention.

It is understood that the choice of these interference particles is made so as to be moreover compatible with the requirements in terms of lightness and saturation required for the compositions according to the invention. In general, these interference particles are present in an amount sufficient to obtain a homogeneous effect in terms of coloration while at the same time preserving the natural flesh tone of the skin.

More specifically, these pigments may be present in amounts ranging from 0.01% to 10% by weight and preferably ranging from 0.1% to 5% by weight relative to the total weight of the composition.

The additional coloring agents may also be selected from fluorescers.

The term “fluorescer” means a substance which, under the effect of ultraviolet rays and/or visible light, re-emits in the visible region the portion of light that it has absorbed under the same color as that which it naturally reflects. The naturally reflected color is thus reinforced by the re-emitted color and appears extremely bright.

Examples include colored polyamide and/or formaldehyde/benzoguanamine and/or melamine/formaldehyde/sulfonamide resins, from colored aminotriazine/formaldehyde/sulfonamide co-condensates and/or from metallized polyester glitter flakes and/or mixtures thereof. These fluorescent pigments may also be present in the form of aqueous dispersions of fluorescent pigments.

Mention may also be made of the pink-colored fluorescent aminotriazine/formaldehyde/sulfonamide co-condensate with a mean particle size of 3-4 microns marketed under the trademark Fiesta Astral Pink FEX-1 and the blue-colored fluorescent aminotriazine/formaldehyde/sulfonamide co-condensate with a mean particle size of 3-4.5 microns marketed under the trademark Fiesta Comet Blue FTX-60 by Swada, or, alternatively, the yellow-colored benzoguanamine/formaldehyde resin covered with formaldehyde/urea resin marketed under the trademark FB-205 Yellow and the red-colored benzoguanamine/formaldehyde resin covered with formaldehyde/urea resin marketed under the trademark FB-400 Orange Red by UK Seung Chemical, and the orange-colored polyamide resin marketed under the trademark Flare 911 Orange 4 by Sterling Industrial Colors.

The fluorescent substances are preferably present in the composition in a content ranging from 0.1% to 20%, preferably from 0.1% to 15% to more preferably from 0.5% to 3% by weight relative to the total weight of the composition.

When the organic fluorescent substances are white, they are also known as optical brighteners.

The optical brightener has the effect of intensifying the radiance and reviving the shades of cosmetic compositions comprising them on application to the skin.

Among the optical brighteners that may be mentioned more particularly are stilbene derivatives, in particular polystyrylstilbenes and triazinestilbenes, coumarin derivatives, in particular hydroxycoumarins and aminocoumarins, oxazole, benzoxazole, imidazole, triazole and pyrazoline derivatives, pyrene derivatives and porphyrin derivatives, and/or mixtures thereof.

Such compounds are available, for example, under the trademarks Tinopal SOP® and Uvitex OB® from the company Ciba Geigy.

The optical brighteners preferentially used are sodium 4,4′-bis[(4,6-dianilino-1,3,5-triazin-2-yl)amino]stilbene-2,2′-disulfonate, 2,5-thiophenediylbis(5-tert-butyl-1,3-benzoxazole) and disodium 4,4′-distyrylbiphenylsulfonate, and/or mixtures thereof.

### Fillers:

The compositions of the invention may comprise at least one filler.

The term “fillers” should be understood to mean colorless or white, mineral or synthetic particles of any form, which are insoluble in the medium of the composition irrespective of the temperature at which the composition is produced. These fillers serve in particular to modify the rheology or the texture of the composition.

The fillers may be mineral or organic and of any form, platelet-shaped, spherical or oblong, irrespective of the crystallographic form (for example lamellar, cubic, hexagonal, orthorhombic, etc.). Exemplary are talc, mica, silica, kaolin, polyamide (Nylon®) powder (Orgasol® from Atochem), poly-β-alanine powder and polyethylene powder, tetrafluoroethylene polymer (Teflon®) powder, lauroyllysine, starch, boron nitride, hollow polymer microspheres such as polyvinylidene chloride/acrylonitrile microspheres, for instance Expancel® (Nobel Industrie), acrylic acid copolymer microspheres (Polytrap® from the company Dow Corning) and silicone resin microbeads (for example Tospearls® from Toshiba), elastomeric polyorganosiloxane particles, precipitated calcium carbonate, magnesium carbonate, magnesium hydrogen carbonate, hydroxyapatite, hollow silica microspheres (Silica Beads® from Maprecos), glass or ceramic microcapsules, and metal soaps derived from organic carboxylic acids having from 8 to 22 carbon atoms and preferably from 12 to 18 carbon atoms, for example zinc stearate, magnesium stearate, lithium stearate, zinc laurate or magnesium myristate.

The compositions according to the invention may in particular comprise at least one matting filler, a soft-focus filler, a fluorescer, an abrasive or exfoliant filler, and mixtures thereof.

### Matting Fillers:

For the purposes of the invention, the term “matting filler” is a spherical or non-spherical, porous or non-porous particle with a refractive index of less than or equal to 2.2, especially less than or equal to 2 and in particular less than or equal to 1.8, preferably ranging from 1.3 to 1.6. The “matting fillers” according to the invention have a volume size comparable to that of the pearlescent agents used. The preferred size of the fillers is thus less than 15 μm measured with a laser granulometer, for instance the Mastersizer 2000® from Malvern or the BI90+ from Brookhaven Instruments Corporation.

In one preferential embodiment of the invention, the “matting fillers” are spherical.

In another preferential embodiment of the invention, the “matting fillers” are porous. In this case, the specific surface area of the particles, which may be related to the porosity, is greater than 10 m²/g and preferably greater than 50 m²/g.

### Mattness Test:

The matting nature of the fillers according to the invention is defined by means of a gonioreflectometer measurement. To do this, the composition containing 5% fillers is spread onto a contrast card (Prufkarte type 24/5—250 cm² marketed by Erichsen) using a mechanical film spreader (wet thickness of 30 μm). The composition is then dried overnight at a temperature of 37° C., and the reflection is then measured using a gonioreflectometer. The result obtained is the ratio R from the specular reflection and the diffuse reflection. The value of R is proportionately smaller the greater the matting effect. The matting fillers according to the invention are those which, at a content of 5% in a cosmetic composition, give a value of R of less than 1 and preferably less than 0.75.

The matting effect of the agent and/or composition containing it may especially be evaluated using a gonioreflectometer, by measuring the ratio R from the specular reflection and the scattered reflection. A value of R of less than or equal to 2 generally reflects a matting effect.

The matting filler may especially be selected from a rice starch or a corn starch, kaolinite, talc, a pumpkin seed extract, cellulose microbeads, plant fibers, synthetic fibers, in particular polyamide fibers, expanded acrylic copolymer microspheres, polyamide powders, silica powders, polytetrafluoroethylene powders, silicone resin powders, acrylic polymer powders, wax powders, polyethylene powders, powders of elastomeric crosslinked organopolysiloxane coated with silicone resin, talc/titanium dioxide/alumina/silica composite powders, amorphous mixed silicate powders, silicate particles and especially mixed silicate particles, and mixtures thereof.

The matting agent may especially be selected from a rice starch or a corn starch, kaolinite, talc, a pumpkin seed extract, cellulose microbeads, plant fibers, synthetic fibers, in particular polyamide fibers, expanded acrylic copolymer microspheres, polyamide powders, silica powders, polytetrafluoroethylene powders, silicone resin powders, acrylic polymer powders, wax powders, polyethylene powders, powders of elastomeric crosslinked organopolysiloxane coated with silicone resin, talc/titanium dioxide/alumina/silica composite powders, amorphous mixed silicate powders, silicate particles and especially mixed silicate particles, and mixtures thereof.

Examples of matting agents that may especially be mentioned include:

rice or corn starch, in particular an aluminum starch octenyl succinate marketed under the trademark Dry Flo® by National Starch;

kaolinite;

silicas;

talc;

a pumpkin seed extract as marketed under the trademark Curbilene® by Indena;

cellulose microbeads as described in EP-1,562,562;

fibers, such as silk fiber, cotton fiber, wool fiber, flax fiber, cellulose fiber extracted especially from wood, from vegetables or from algae, polyamide fiber (Nylon®), modified cellulose fiber, poly-p-phenyleneterephthamide fiber, acrylic fiber, polyolefin fiber, glass fiber, silica fiber, aramid fiber, carbon fiber, Teflon® fiber, insoluble collagen fiber, polyester fiber, polyvinyl chloride or polyvinylidene chloride fiber, polyvinyl alcohol fiber, polyacrylonitrile fiber, chitosan fiber, polyurethane fiber, polyethylene phthalate fiber, fibers formed from a mixture of polymers, resorbable synthetic fibers, and mixtures thereof described in EP-1,151,742;

expanded acrylic copolymer microspheres such as those marketed by EXPANCEL under the trademark Expancel 551®;

fillers with an optical effect as described in FR-2,869,796, in particular:

polyamide powders (Nylon®), for instance Nylon 12 particles of the Orgasol type from Arkema, with a mean size of 10 microns and a refractive index of 1.54,

silica powders, for instance Silica beads SB150 from Miyoshi with a mean size of 5 microns and a refractive index of 1.45,

polytetrafluoroethylene powders, for instance PTFE Ceridust 9205F from Clariant, with a mean size of 8 microns and a refractive index of 1.36,

silicone resin powders, for instance the silicone resin Tospearl 145A from GE Silicone with a mean size of 4.5 microns and a refractive index of 1.41,

acrylic copolymer powders, especially of polymethyl(meth)acrylate, for instance the PMMA particles Jurymer MBI from Nihon Junyoki, with a mean size of 8 microns and a refractive index of 1.49, or the Micropearl M100® and F 80 ED® particles from the company Matsumoto Yushi-Seiyaku,

wax powders, for instance the paraffin wax particles Microease 114S from Micropowders, with a mean size of 7 microns and a refractive index of 1.54,

polyethylene powders, especially comprising at least one ethylene/acrylic acid copolymer, and in particular constituted of ethylene/acrylic acid copolymers, for instance the particles Flobeads EA 209 from Sumitomo (with a mean size of 10 microns and a refractive index of 1.48),

elastomeric crosslinked organopolysiloxane powders coated with silicone resin, especially with silsesquioxane resin, as described, for example, in U.S. Pat. No. 5,538,793. Such elastomeric powders are marketed under the trademarks KSP-100, KSP-101, KSP-102, KSP-103, KSP-104 and KSP-105 by Shin-Etsu, and

talc/titanium dioxide/alumina/silica composite powders such as those marketed under the trademark Coverleaf® AR-80 by Catalyst & Chemicals,

mixtures thereof,

compounds that absorb and/or adsorb sebum as described in FR-2,869,796. Especially exemplary are:

silica powders, for instance the porous silica microspheres marketed under the trademark Silica Beads SB-700 marketed by Myoshi, the products Sunsphere® H51, Sunsphere® H33 and Sunsphere® H53 marketed by Asahi Glass; the polydimethylsiloxane-coated amorphous silica microspheres marketed under the trademark SA Sunsphere® H-33 and SA Sunsphere® H-53 marketed by Asahi Glass;

amorphous mixed silicate powders, especially of aluminum and magnesium, for instance the product marketed under the trademark Neusilin UFL2 by Sumitomo;

polyamide (Nylon®) powders, for instance Orgasol® 4000 marketed by Arkema, and

acrylic polymer powders, especially of polymethyl methacrylate, for instance Covabead® LH85 marketed by Wacker; of polymethyl methacrylate/ethylene glycol dimethacrylate, for instance Dow Corning 5640 Microsponge® Skin Oil Adsorber marketed by Dow Corning, or Ganzpearl® GMP-0820 marketed by Ganz Chemical; of polyallyl methacrylate/ethylene glycol dimethacrylate, for instance Poly-Pore® L200 or Poly-Pore® E200 marketed by Amcol; of ethylene glycol dimethacrylate/lauryl methacrylate copolymer, for instance Polytrap® 6603 marketed by Dow Corning;

silicate particles, such as alumina silicate;

mixed silicate particles, such as:

magnesium aluminum silicate particles, such as saponite or hydrated magnesium aluminum silicate with a sodium sulfate marketed under the trademark Sumecton® by Kunimine;

the magnesium silicate, hydroxyethylcellulose, black cumin oil, marrow oil and phospholipids complex or Matipure® from Lucas Meyer, and

mixtures thereof.

Preferred matting agents that may be used according to the invention include a pumpkin seed extract, a rice or corn starch, kaolinite, silicas, talc, polyamide powders, polyethylene powders, acrylic copolymer powders, expanded acrylic copolymer microspheres, silicone resin microbeads and mixed silicate particles, and mixtures thereof.

Fillers with a Soft-Focus Effect:

These fillers may be any material capable of modifying and hiding wrinkles by virtue of their intrinsic physical properties. These fillers may especially modify wrinkles via a tensioning effect, a covering effect or a soft-focus effect.

Examples of fillers that may be given include the following compounds:

porous silica microparticles, for instance the Silica Beads® SB150 and SB700 from Miyoshi with a mean size of 5 μm; the series-H Sunspheres® from Asahi Glass, for instance Sunspheres H33, H51 with respective sizes of 3.5 and 5 μm;

hollow hemispherical silicone resin particles such as NLK 500®, NLK 506® and NLK 510® from Takemoto Oil and Fat, especially described in EP-A-1 579 849;

silicone resin powders, for instance the silicone resin Tospearl® 145A from GE Silicone, with a mean size of 4.5 μm;

acrylic copolymer powders, especially of polymethyl (meth)acrylate, for instance the PMMA particles Jurimer MBI® from Nihon Junyoki, with a mean size of 8 μm, the hollow PMMA spheres marketed under the trademark Covabead® LH85 by Wacker, and vinylidene/acrylonitrile/methylene methacrylate expanded microspheres marketed under the trademark Expancel®;

wax powders, for instance the paraffin wax particles MicroEase® 114S from MicroPowders, with a mean size of 7 μm;

polyethylene powders, especially comprising at least one ethylene/acrylic acid copolymer for instance the Flobeads® EA 209 E from Sumitomo, with a mean size of 10 μm;

crosslinked elastomeric organopolysiloxane powders coated with silicone resin and especially with silsesquioxane resin, under the trademarks KSP-100®, KSP-101®, KSP-102®, KSP-103®, KSP-104® and KSP-105® by Shin-Etsu;

talc/titanium dioxide/alumina/silica composite powders, for instance those marketed under the trademark Coverleaf AR-80® by Catalysts & Chemicals;

talc, mica, kaolin, lauryl glycine, starch powders crosslinked with octenyl succinate anhydride, boron nitride, polytetrafluoroethylene powders, precipitated calcium carbonate, magnesium carbonate, magnesium hydrogen carbonate, barium sulfate, hydroxyapatite, calcium silicate, cerium dioxide and glass or ceramic microcapsules;

hydrophilic or hydrophobic, synthetic or natural, mineral or organic fibers such as silk fibers, cotton fibers, wool fibers, flax fibers, cellulose fibers extracted especially from wood, vegetables or algae, polyamide (Nylon®) fibers, modified cellulose fibers, poly-p-phenyleneterephthamide fibers, acrylic fibers, polyolefin fibers, glass fibers, silica fibers, aramid fibers, carbon fibers, polytetrafluoroethylene (Teflon®) fibers, insoluble collagen fibers, polyester fibers, polyvinyl chloride fibers, polyvinylidene chloride fibers, polyvinyl alcohol fibers, polyacrylonitrile fibers, chitosan fibers, polyurethane fibers, polyethylene phthalate fibers, fibers formed from a mixture of polymers, resorbable synthetic fibers, and mixtures thereof described in EP-1,151,742;

spherical elastomeric crosslinked silicones, for instance Trefil E-505C® or E-506C® from Dow Corning;

abrasive fillers, which, via a mechanical effect, smooth out the skin microrelief, such as abrasive silica, for instance Abrasif SP® from Semanez or nut or shell powders (for example of apricot or walnut, from Cosmetochem).

The fillers with an effect on the signs of aging are especially selected from porous silica microparticles, hollow hemispherical silicone particles, silicone resin powders, acrylic copolymer powders, polyethylene powders, crosslinked elastomeric organopolysiloxane powders coated with silicone resin, talc/titanium dioxide/alumina/silica composite powders, precipitated calcium carbonate, magnesium carbonate, magnesium hydrogen carbonate, barium sulfate, hydroxyapatite, calcium silicate, cerium dioxide, glass or ceramic microcapsules, and silk fibers or cotton fibers, and mixtures thereof.

The filler may be a soft-focus filler.

The term “soft-focus” filler means a filler which in addition gives the complexion transparency and a hazy effect. Preferably, the “soft-focus” fillers have a mean particle size of less than or equal to 15 microns. These particles may be in any form and in particular may be spherical or non-spherical. These fillers are more preferably non-spherical.

The “soft-focus” fillers may be selected from silica and silicate powders, especially alumina powder, powders of polymethyl methacrylate (PMMA) type, talc, silica/TiO₂ or silica/zinc oxide composites, polyethylene powders, starch powders, polyamide powders, styrene/acrylic copolymer powders and silicone elastomers, and mixtures thereof.

Exemplary are, in particular, talc with a number-average size of less than or equal to 3 microns, for example talc with a number-average size of 1.8 microns and especially the product marketed under the trademark Talc P3® by Nippon Talc, Nylon® 12 powder, especially the product marketed under the trademark Orgasol 2002 Extra D Nat Cos® by Atochem, silica particles 1% to 2% surface-treated with a mineral wax (INCI name: hydrated silica (and) paraffin) such as the products marketed by Degussa, amorphous silica microspheres, such as the products marketed under the trademark Sunsphere, for example of reference H-53® by Asahi Glass, and silica microbeads such as those marketed under the trademark SB-700® or SB-150® by Miyoshi, this list not being limiting.

The concentration of these fillers with an effect on the signs of aging in the compositions according to the invention may be from 0.1% to 40%, or even from 0.1% to 20% by weight, relative to the total weight of the composition.

### Abrasive Fillers or Exfoliants:

As exfoliants that may be used in rinse-out compositions according to the invention, examples include exfoliants or scrubbing particles of mineral, plant or organic origin. Thus, polyethylene beads or powder, Nylon powder, polyvinyl chloride powder, pumice powder, ground apricot kernel or walnut husk, sawdust, glass beads and alumina, and mixtures thereof, may be used, for example.

Mention may also be made of Exfogreen® from Solabia (bamboo extract), extracts of strawberry akenes (Strawberry Akenes from Greentech), peach kernel powder, apricot kernel powder, and finally, in the field of plant powders with an abrasive effect, exemplary are cranberry kernel powder.

As abrasive fillers or exfoliants that are preferred according to the invention, mention will be made of peach kernel powder, apricot kernel powder, cranberry kernel powder, strawberry akene extracts and bamboo extracts.

The additional filler(s) used in the compositions according to the invention may represent preferably from 0.01% to 20% by weight and better still from 0.1% to 15%, and even better still, from 0.5 to 5% by weight relative to the total weight of the composition.

### Cosmetic or Dermatological Active Agents:

The compositions according to the invention may comprise one or more additional cosmetic or dermatological active agents.

The additional active agents may in particular be selected from moisturizers, desquamating agents, agents for improving the barrier function, depigmenting agents, antioxidants, dermo-decontracting agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting the maturation of the horny envelope, NO-synthase inhibitors, peripheral benzodiazepine receptor (PBR) antagonists, agents for increasing the activity of the sebaceous glands, agents for stimulating the energy metabolism of cells, tensioning agents, fat-restructuring agents, slimming agents, agents for promoting the cutaneous capillary circulation, calmatives and/or anti-irritants, sebo-regulators or anti-seborrheic agents, astringents, cicatrizing agents, anti-inflammatory agents and antiacne agents.

Those skilled in the art will select said active agent(s) as a function of the effect desired on the skin, the lips, the nails, the eyelashes or the eyebrows.

Of course, those skilled in the art will take care to select this or these optional additional compound(s), and/or the amount thereof, in such a way that the advantageous properties of the corresponding composition according to the invention are not, or are not substantially, impaired by the envisaged addition.

For caring for and/or making up aged skin, they will preferably choose at least one active agent selected from moisturizers, desquamating agents, agents for improving the barrier function, depigmenting agents, antioxidants, dermo-decontracting agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting the maturation of the horny envelope, NO-synthase inhibitors, peripheral benzodiazepine receptor (PBR) antagonists, agents for increasing the activity of the sebaceous glands, agents for stimulating the energy metabolism of cells, fat-restructuring agents and agents for promoting the cutaneous capillary circulation for the area around the eyes.

For caring for and/or making up greasy skin, those skilled in the art will preferably choose at least one active agent selected from desquamating agents, sebo-regulating agents or anti-seborrheic agents, and astringents.

For caring for and/or making up acne-prone skin, they will preferably choose at least one active agent selected from antiacne agents, cicatrizing agents and anti-inflammatory agents.

For slimming care of the body, they will preferably choose an active agent selected from slimming agents and active agents for promoting the cutaneous capillary circulation.

Examples of such compounds are described hereinafter.

### Moisturizers or Humectants:

Moisturizers or humectants that may especially be mentioned include glycerol and derivatives thereof, urea and derivatives thereof, especially Hydrovance® marketed by National Starch, lactic acid, hyaluronic acid, AHAs, BHAs, sodium pidolate, xylitol, serine, sodium lactate, ectoin and derivatives thereof, chitosan and derivatives thereof, collagen, plankton, an extract of Imperata cylindra marketed under the trademark Moist 24® by Sederma, acrylic acid homopolymers, for instance Lipidure-HM® from NOF Corporation, beta-glucan and in particular sodium carboxymethyl beta-glucan from Mibelle-AG-Biochemistry; a mixture of passionflower oil, apricot oil, corn oil and rice bran oil marketed by Nestlé under the trademark NutraLipids®; a C-glycoside derivative such as those described in WO 02/051 828 and in particular C-β-D-xylopyranoside-2-hydroxypropane in the form of a solution containing 30% by weight of active material in a water/propylene glycol mixture (60/40% by weight) such as the product marketed by Chimex under the trademark Mexoryl SBB®; an oil of musk rose marketed by Nestlé; an extract of the microalga Prophyridium cruentum enriched with zinc, marketed by Vincience under the trademark Algualane Zinc®; spheres of collagen and of chondroitin sulfate of marine origin (Atelocollagen) marketed by Engelhard Lyon under the trademark Marine Filling Spheres; hyaluronic acid spheres such as those marketed by Engelhard Lyon.

The moisturizer that will preferably be used is selected from urea and derivatives thereof, especially Hydrovance® marketed by National Starch, hyaluronic acid, AHAs, BHAs, acrylic acid homopolymers, for instance Lipidure-HM® from NOF Corporation, beta-glucan and in particular sodium carboxymethyl beta-glucan from Mibelle-AG-Biochemistry; a mixture of passionflower oil, apricot oil, corn oil and rice bran oil marketed by Nestlé under the trademark NutraLipids®; a C-glycoside derivative such as those described in WO 02/051 828 and in particular C-β-D-xylopyranoside-2-hydroxypropane in the form of a solution containing 30% by weight of active material in a water/propylene glycol mixture (60/40% by weight) such as the product marketed by Chimex under the trademark Mexoryl SBB®; an oil of musk rose marketed by Nestlé; an extract of the microalga Prophyridium cruentum enriched with zinc, marketed by Vincience under the trademark Algualane Zinc®; spheres of collagen and of chondroitin sulfate of marine origin (Atelocollagen) marketed by Engelhard Lyon under the trademark Marine Filling Spheres; hyaluronic acid spheres such as those marketed by Engelhard Lyon.

### 2. Desquamating Agents:

The term “desquamating agent” means any compound capable of acting:

either directly on desquamation by promoting exfoliation, such as β-hydroxy acids (BHAs), in particular salicylic acid and derivatives thereof (including 5-n-octanoylsalicylic acid, also known as capryloyl salicylic acid as the INCI name); α-hydroxy acids (AHAs), such as glycolic acid, citric acid, lactic acid, tartaric acid, malic acid or mandelic acid; 8-hexadecene-1,16-dicarboxylic acid or 9-octadecenedioic acid; urea and derivatives thereof; gentisic acid and derivatives thereof; oligofucoses; cinnamic acid; Saphora japonica extract; resveratrol, and certain jasmonic acid derivatives;

or on the enzymes involved in the desquamation or degradation of corneodesmosomes, glycosidases, stratum corneum chymotryptic enzyme (SCCE) or other proteases (trypsin, chymotrypsin-like). Exemplary are aminosulfonic compounds and in particular 4-(2-hydroxyethyl)piperazine-1-propanesulfonic acid (HEPES); 2-oxothiazolidine-4-carboxylic acid (procysteine) and derivatives thereof; derivatives of α-amino acids of glycine type (as described in EP-0 852 949, and also sodium methyl glycine diacetate marketed by BASF under the trademark Trilon M); honey; sugar derivatives such as O-octanoyl-6-D-maltose and N-acetylglucosamine. As other desquamating agents that may be used in the composition according to the invention, exemplary are:

oligofructoses, EDTA and derivatives thereof, laminaria extracts, O-linoleyl-6D-glucose, (3-hydroxy-2-pentylcyclopentyl)acetic acid, glycerol trilactate, O-octanyl-6′-D-maltose, S-carboxymethylcysteine, siliceous derivatives of salicylate such as those described in EP-0,796,861, oligofucases such as those described in EP-0,218,200,5-acyl salicylic acid salts, active agents with effects on transglutaminase, as in EP-0,899,330,

extract of the flowers of ficus Opuntia indica (Exfolactive® from Silab),

8-hexadecene-1,16-dicarboxylic acid,

esters of glucose and of vitamin F, and

mixtures thereof.

Preferred desquamating agents include β-hydroxy acids such as 5-n-octanoyl salicylic acid; urea; glycolic acid, citric acid, lactic acid, tartaric acid, malic acid or mandelic acid; 4-(2-hydroxyethyl)piperazine-1-propanesulfonic acid (HEPES); extract of Saphora japonica; honey; N-acetyl glucosamine; sodium methyl glycine diacetate, and mixtures thereof.

Even more preferentially, a desquamating agent selected from 5-n-octanoyl salicylic acid; urea; 4-(2-hydroxyethyl)piperazine-1-propanesulfonic acid (HEPES); extract of Saphora japonica; honey; N-acetyl glucosamine; sodium methyl glycine diacetate, and mixtures thereof, will be used in the compositions of the invention.

### 3. Agents for Improving the Barrier Function:

As agents for improving the barrier function, especially exemplary are arginine, serine, an extract of Thermus thermophilus such as Venuceane® from Sederma, an extract of the rhizome of wild yam (Dioscorea villosa) such as Actigen Y® from Active Organics, plankton extracts, for instance Omega Plankton® from Secma, yeast extracts, for instance Relipidium® from Coletica, a chestnut extract such as Recoverine® from Silab, a cedar bud extract such as Gatuline Zen® from Gattefossé, sphingosines, for instance salicyloyl sphingosine marketed under the trademark Phytosphingosine® SLC by Degussa, a mixture of xylitol, polyxylityl glycoside and xylitan, for instance Aquaxyl® from SEPPIC, extracts of Solanacea plants, for instance Lipidessence® from Coletica, omega-3 unsaturated oils such as musk rose oils, and mixtures thereof.

Mention may also be made especially of ceramides or derivatives thereof, in particular ceramides of type 2 (for instance N-oleoyldihydrosphingosine), of type 3 (for instance stearoyl-4-hydroxysphinganine, as the INCI name) and of type 5 (for instance N-2-hydroxypalmitoyldihydrosphingosine, having the INCI name: hydroxypalmitoyl sphinganine), sphingoid-based compounds, glycosphingolipids, phospholipids, cholesterol and derivatives thereof, phytosterols, essential fatty acids, diacylglycerol, 4-chromanone and chromone derivatives, petroleum jelly, lanolin, shea butter, cocoa butter and PCA salts.

As preferred agents having a restructuring effect on the barrier function, mention will be made of an extract of Thermus thermophilus, an extract of wild yam rhizome (Dioscorea villosa), a yeast extract, a chestnut extract, a cedar bud extract, arginine, serine, ceramides especially of type 3 and 5; and mixtures thereof.

Serine, arginine or a mixture thereof will preferably be used.

### 4. Depigmenting Agents:

Depigmenting agents that may especially be mentioned include alpha- and beta-arbutin, ferulic acid, lucinol and derivatives thereof, kojic acid, resorcinol and derivatives thereof, tranexamic acid and derivatives thereof, gentisic acid, homogentisate, methyl gentisate or homogentisate, dioic acid, calcium D-pantheteine sulfonate, lipoic acid, ellagic acid, vitamin B3, linoleic acid and derivatives thereof, ceramides and homologues thereof, plant derivatives, for instance camomile, bearberry, the aloe family (vera, ferox, bardensis), mulberry or skullcap; a kiwi fruit (Actinidia chinensis) juice marketed by Gattefosse, an extract of Paeonia suffruticosa root, such as the product marketed by Ichimaru Pharcos under the trademark Botanpi Liquid B®, an extract of brown sugar (Saccharum officinarum), such as the extract of molasses marketed by Taiyo Kagaku under the trademark Molasses Liquid, without this list being exhaustive.

Preferred depigmenting agents that will be used include alpha- and beta-arbutin, ferulic acid, kojic acid, resorcinol and derivatives thereof, calcium D-pantheteine sulfonate, lipoic acid, ellagic acid, vitamin B3, a kiwi fruit (Actinidia chinensis) juice marketed by Gattefosse, and an extract of Paeonia suffruticosa root, such as the product marketed by Ichimaru Pharcos under the trademark Botanpi Liquid B®.

### 5. Antioxidants:

Exemplary are tocopherol and esters thereof, in particular tocopheryl acetate; ferulic acid; serine; ellagic acid, phloretin, polyphenols, tannins, tannic acid, epigallocatechins and natural extracts containing them, anthocyans, rosemary extracts, olive leaf extracts, for instance those from the company Silab, green tea extracts, resveratrol and derivatives thereof, ergothioneine, N-acetylcysteine, an extract of the brown alga Pelvetia caniculata, for instance Pelvetiane® from Secma, chlorogenic acid, biotin, chelating agents, such as BHT and BHA, N,N′-bis(3,4,5-trimethoxybenzyl)ethylenediamine and salts thereof; idebenone, plant extracts, for instance Pronalen Bioprotect™ from the company Provital; coenzyme Q10, bioflavonoids, SODs, phytanetriol, lignans, melatonin, pidolates, glutathione, caprylyl glycol, Totarol™ or extract of Podocarpus totara containing Totarol (totara-8,11,13-trienol or 2-phenanthrenol, 4b,5,6,7,8,8a,9,10-octahydro-4b,8,8-trimethyl-1-(1-methylethyl)-; a jasmine extract such as the product marketed by Silab under the trademark Helisun®; hesperitin laurate such as Flavagrum PEG® from the company Engelhard Lyon; an extract of Paeonia suffruticosa root, such as the product marketed by Ichimaru Pharcos under the trademark Botanpi Liquid B®; an extract of lychee such as the extract of lychee pericarp marketed by Cognis under the trademark Litchiderm LS 9704®, an extract of pomegranate fruit (Punica granatum), such as the product marketed by Draco Natural Products.

Other anti-aging agents that are exemplary include DHEA and derivatives thereof, boswellic acid, rosemary extracts, carotenoids (β-carotene, zeaxanthin and lutein), cysteic acid, copper derivatives and jasmonic acid.

Preferred antioxidants that will especially be used include ferulic acid; serine; phloretin, an extract of pomegranate, biotin, chelating agents such as BHT, BHA, N,N′-bis(3,4,5-trimethoxybenzyl)ethylenediamine and salts thereof, caprylyl glycol, Totarol™, a jasmine extract such as the product marketed by Silab under the trademark Helisun®; hesperitin laurate such as Flavagrum PEG® from the company Engelhard Lyon; an extract of Paeonia suffruticosa root, such as the product marketed by Ichimaru Pharcos under the trademark Botanpi Liquid B®.

### 6. Dermo-Relaxing or Dermo-Decontracting Agents:

Examples include manganese gluconate and other salts, adenosine, alverine citrate and salts thereof, glycine, an extract of Iris pallida, a hexapeptide (Argeriline R from Lipotec) or sapogenins, for instance wild yam and the carbonyl amines described in EP-1,484,052. Examples of sapogenins that are exemplary include those described in WO 02/47650, in particular wild yam, the diosgenin extracted especially from Dioscorea opposita or any extract naturally containing or containing after treatment one or more sapogenins (wild yam rhizome, agave leaf, which contains hecogenin and tigogenin, extracts of Liliacea plants and more particularly yucca or smilax containing smilagenin and sarsapogenin, or sarsaparilla) or Actigen Y from the company Active Organics, or ginger.

Mention may also be made of DMAE (dimethyl MEA), extracts of sea fennel, of rockrose, of helichrysum, of aniseed, of paracress, and an extract of Acmella oleracea, for instance Gatuline® from Gattefossé.

Preferred dermo-relaxing agents that will be mentioned include adenosine, manganese gluconate, wild yam, sea fennel, glycine and alverine.

### 7. Anti-Glycation Agents:

The term “anti-glycation agent” means a compound that prevents and/or reduces the glycation of skin proteins, in particular dermal proteins such as collagen.

Anti-glycation agents that may especially be mentioned include extracts of plants of the Ericacea family, such as an extract of blueberry (Vaccinium angustifolium or Vaccinium myrtillus), for example the product marketed under the trademark Blueberry Herbasol Extract PG by Cosmetochem, ergothioneine and derivatives thereof, hydroxystilbenes and derivatives thereof, such as resveratrol and 3,3′,5,5′-tetrahydroxystilbene (these anti-glycation agents are described in FR-2,802,425, FR-2,810,548, FR-2,796,278 and FR-2,802,420, respectively), dihydroxystilbenes and derivatives thereof, polypeptides of arginine and of lysine such as the product marketed under the trademark Amadorine® by Solabia, carsinine hydrochloride (marketed by Exsymol under the trademark Alistin®), an extract of Helianthus annuus, for instance Antiglyskin® from Silab, wine extracts such as the extract of powdered white wine on a maltodextrin support marketed under the trademark Vin blanc déshydraté 2F by Givaudan, thioctic acid (or alpha-lipoic acid), a mixture of extract of bearberry and of marine glycogen, for instance Aglycal LS 8777® from Laboratoires Sérobiologiques, and an extract of black tea, for instance Kombuchka® from Sederma, and mixtures thereof.

Preferred anti-glycation agents that will be mentioned include extracts of blueberry (Vaccinium myrtillus) and extracts of black tea.

8. Agents for Stimulating the Synthesis of Dermal and/or Epidermal Macromolecules and/or for Preventing their Degradation:

Among the active agents for stimulating the dermal macromolecules or for preventing their degradation, exemplary are those acting:

either on collagen synthesis, such as extracts of Centella asiatica, asiaticosides and derivatives thereof; ascorbic acid or vitamin C and derivatives thereof; synthetic peptides such as iamin, biopeptide CL or palmitoyl oligopeptide marketed by Sederma; peptides extracted from plants, such as the soybean hydrolysate marketed by Coletica under the trademark Phytokine®; rice peptides such as Nutripeptide® from Silab, methylsilanol mannuronate such as Algisium C® marketed by Exsymol; plant hormones such as auxins and lignans; folic acid; and an extract of Medicago sativa (alfalfa) such as the product marketed by Silab under the trademark Vitanol®; a peptide extract of hazelnut such as the product marketed by Solabia under the trademark Nuteline C®; and arginine;

or on the inhibition of collagen degradation, in particular agents acting on the inhibition of metalloproteases (MMP) more particularly such as MMP 1, 2, 3 and 9. Exemplary are: retinoids and derivatives, extracts of Medicago sativa such as Vitanol® from Silab, an extract of Aphanizomenon flos-aquae (Cyanophyceae) marketed under the trademark Lanablue® by Atrium Biotechnologies, oligopeptides and lipopeptides, lipoamino acids, the malt extract marketed by Coletica under the trademark Collalift®; blueberry or rosemary extracts; lycopene; isoflavones, derivatives thereof or plant extracts containing them, in particular extracts of soybean (marketed, for example, by Ichimaru Pharcos under the trademark Flavosterone SB®), of red clover, of flax or of kakkon; an extract of lychee such as the extract of lychee pericarp marketed by Cognis under the trademark Litchiderm LS 9704®; Dipalmitoyl Hydroxyproline marketed by SEPPIC under the trademark Sepilift DPHP®: Baccharis genistelloides or Baccharine marketed by Silab, an extract of moringa such as Arganyl LS 9781® from Cognis; the sage extract described in FR-A-2,812,544 from the Labiatae family (Salvia officinalis from the company Flacksmann), an extract of rhododendron, a blueberry extract, and an extract of Vaccinium myrtillus such as those described in FR-A-2,814,950;

or on the synthesis of molecules belonging to the elastin family (elastin and fibrillin), such as: retinol and derivatives, in particular retinyl palmitate; the extract of Saccharomyces cerevisiae marketed by LSN under the trademark Cytovitin®; and the extract of the alga Macrocystis pyrifera marketed by Secma under the trademark Kelpadelie®; a peptide extract of hazelnut such as the product marketed by Solabia under the trademark Nuteline C®;

or on inhibition of elastin degradation, such as the peptide extract of seeds of Pisum sativum marketed by LSN under the trademark Parelastyl®; heparinoids; and the N-acylamino acid compounds described in WO 01/94381, such as {2-[acetyl(3-trifluoromethylphenyl)amino]-3-methylbutyrylamino}acetic acid, also known as N—[N-acetyl, N′-(3-trifluoromethyl)phenylvalyl]glycine, or N-acetyl-N-[3-(trifluoromethyl)phenyl]valylglycine or acetyl trifluoromethylphenylvalylglycine, or an ester thereof with a C₁-C₆ alcohol; an extract of rice peptides such as Colhibin® from Pentapharm, or an extract of Phyllanthus emblica such as Emblica® from Rona;

or on the synthesis of glycosaminoglycans, such as the product of fermentation of milk with Lactobacillus vulgaris, marketed by Brooks under the trademark Biomin Yoghurt®; the extract of the brown alga Padina pavonica marketed by Alban Müller under the trademark HSP3®; the Saccharomyces cerevisiae extract available especially from the company Silab under the trademark Firmalift® or from the company LSN under the trademark Cytovitin®; an extract of Laminaria ochroleuca such as Laminaine® from Secma; essence of Mamaku from Lucas Meyer, and an extract of Cress (Odraline® from Silab);

or on the synthesis of fibronectin, such as the extract of the zooplankton Salina marketed by Seporga under the trademark GP4G®; the yeast extract available especially from the company Alban Müller under the trademark Drieline®; and the palmitoyl pentapeptide marketed by Sederma under the trademark Matrixyl®.

Among the active agents for stimulating epidermal macromolecules, such as fillagrin and keratins, especially exemplary are the extract of lupin marketed by Silab under the trademark Structurine®; the extract of Fagus sylvatica beech buds marketed by Gattefosse under the trademark Gatuline® RC; and the extract of the zooplankton Salina marketed by Seporga under the trademark GP4G®; the copper tripeptide from Procyte; a peptide extract of Voandzeia substerranea such as the product marketed by Laboratoires Sérobiologiques under the trademark Filladyn LS 9397®.

Preferably, an active agent that stimulates the synthesis of dermal and/or epidermal macromolecules and/or that prevents their degradation, selected from agents for stimulating the synthesis of glycosaminoglycans, agents for inhibiting elastin degradation, agents for stimulating fibronectin synthesis, agents for stimulating the synthesis of epidermal macromolecules, and mixtures thereof, will be used.

Even more preferentially, an active agent that stimulates the synthesis of the glycosaminoglycans, selected from an extract of the brown alga Padina pavonica, an extract of Saccharomyces cerevisiae, an extract of Laminaria ochroleuca, essence of Mamaku, and an extract of cress, and mixtures thereof, will be used.

As preferred active agents for stimulating the synthesis of dermal and/or epidermal macromolecules and/or for preventing their degradation, exemplary are:

synthetic peptides such as iamin, the biopeptide CL or palmitoyloligopeptide marketed by Sederma; peptides extracted from plants, such as the soybean hydrolysate marketed by Coletica under the trademark Phytokine®; rice peptides such as Nutripeptide® from Silab, methylsilanol mannuronate such as Algisium C® marketed by Exsymol; folic acid; an extract of Medicago sativa (alfalfa), such as the product marketed by Silab under the trademark Vitanol®; a peptide extract of hazelnut, such as the product marketed by Solabia under the trademark Nuteline C®; arginine; an extract of Aphanizomenon flos-aquae (Cyanophyceae) marketed under the trademark Lanablue® by Atrium Biotechnologies, the malt extract marketed by Coletica under the trademark Collalift®, lycopene; an extract of lychee; an extract of moringa such as Arganyl LS 9781® from Cognis; an extract of Vaccinium myrtillus such as those described in FR-A-2,814,950; retinol and derivatives thereof, in particular retinyl palmitate; the extract of Saccharomyces cerevisiae marketed by LSN under the trademark Cytovitin®; a peptide extract of hazelnut such as the product marketed by Solabia under the trademark Nuteline C®; {2-[acetyl(3-trifluoromethylphenyl)amino]-3-methylbutyrylamino}acetic acid, also known as N—[N-acetyl, N′-(3-trifluoromethyl)phenylvalyl]glycine, or N-acetyl-N-[3-(trifluoromethyl)phenyl]valylglycine or acetyl trifluoromethylphenylvalylglycine, or an ester thereof with a C₁-Cₛ alcohol; an extract of rice peptides such as Colhibin® from Pentapharm, or an extract of Phyllanthus emblica such as Emblica® from Rona; the extract of the brown alga Padina pavonica marketed by Alban Müller under the trademark HSP3®; the extract of Saccharomyces cerevisiae available especially from the company Silab under the trademark Firmalift® or from the company LSN under the trademark Cytovitin®; an extract of Laminaria ochroleuca such as Laminaine® from Secma; the essence of Mamaku from Lucas Meyer, the extract of lupin marketed by Silab under the trademark Structurine®; the extract of Fagus sylvatica beech buds marketed by Gattefosse under the trademark Gatuline® RC.

9. Agents for Stimulating Fibroblast or Keratinocyte Proliferation and/or Keratinocyte Differentiation:

The agents for stimulating fibroblast proliferation that may be included in the compositions according to the invention may be selected, for example, from plant proteins or polypeptides, extracted especially from soybean (for example a soybean extract marketed by LSN under the trademark Eleseryl SH-VEG 8® or marketed by Silab under the trademark Raffermine®); an extract of hydrolysed soybean proteins such as Ridulisse® from Silab; and plant hormones such as gibberellins and cytokinins; a peptide extract of hazelnut such as the product marketed by Solabia under the trademark Nuteline C®.

Preferably, an agent that promotes keratinocyte proliferation and/or differentiation will be used.

The agents for stimulating keratinocyte proliferation that may be used in the composition according to the invention especially comprise adenosine; phloroglucinol, the extracts of Hydrangea macrophylla leaves, for instance Amacha Liquid E® from Ichimaru Pharcos, a yeast extract such as Stimoderm® from CLR; the extract of Larrea divaricata such as Capislow® from Sederma, mixtures of extracts of papaya, of olive leaves and of lemon, such as Xyleine® from Vincience, retinol and esters thereof, including retinyl palmitate, phloroglucinol, the nut cake extracts marketed by the Gattefosse and the extracts of Solanum tuberosum such as Dermolectine® marketed by Sederma.

Among the agents for stimulating keratinocyte differentiation are, for example, minerals such as calcium; sea fennel, a peptide extract of lupin, such as the product marketed by Silab under the trademark Structurine®; sodium beta-sitosteryl sulfate, such as the product marketed by Seporga under the trademark Phytocohesine®; and a water-soluble extract of corn, such as the product marketed by Solabia under the trademark Phytovityl®; a peptide extract of Voandzeia substerranea such as the product marketed by Laboratoires Sérobiologiques under the trademark Filladyn LS 9397®; and lignans such as secoisolariciresinol, and retinol and esters thereof, including retinyl palmitate.

As agents for stimulating keratinocyte proliferation and/or differentiation, mention may also be made of oestrogens such as oestradiol and homologues; cytokines.

As preferred active agents for stimulating fibroblast or keratinocyte proliferation and/or keratinocyte differentiation, mention will be made of plant proteins or polypeptides, extracted especially from soybean (for example a soybean extract marketed by LSN under the trademark Eleseryl SH-VEG 8® or marketed by Silab under the trademark Raffermine®); an extract of hydrolysed soybean proteins such as Ridulisse® from Silab; a peptide extract of hazelnut such as the product marketed by Solabia under the trademark Nuteline C®; adenosine; phloroglucinol, a yeast extract such as Stimoderm® from CLR; a peptide extract of lupin such as the product marketed by Silab under the trademark Structurine®; a water-soluble corn extract, such as the product marketed by Solabia under the trademark Phytovityl®; a peptide extract of Voandzeia substerranea, such as the product marketed by Laboratoires Sérobiologiques under the trademark Filladyn LS 9397®; retinol and esters thereof, including retinyl palmitate.

### 10. Agents for Promoting the Maturation of the Horny Envelope:

Agents that participate in the maturation of the horny envelope, which becomes impaired with age and induces a decrease in transglutaminase activity, may be included in the compositions of the invention. Examples include urea and derivatives thereof and in particular Hydrovance® from National Starch and the other active agents mentioned in L'Oreal FR-2,877,220 (unpublished).

### 11. NO-Synthase Inhibitors:

The agent with an inhibitory action on NO synthase may be selected from OPCs (procyanidol oligomers); plant extracts of the species Vitis vinifera marketed especially by Euromed under the trademark “Leucocyanidines de raisins extra”, or by Indena under the trademark Leucoselect®, or finally by Hansen under the trademark “Extrait de marc de raisin”; plant extracts of the species Olea europaea preferably obtained from olive tree leaves and marketed especially by Vinyals in the form of a dry extract, or by Biologia & Technologia under the trademark Eurol® BT; and plant extracts of the species Gingko biloba, preferably a dry aqueous extract of this plant marketed by Beaufour under the trademark “Ginkgo biloba extrait standard”, and mixtures thereof.

### 12. Peripheral Benzodiazepine Receptor (PBR) Antagonists:

Exemplary are, for example, 1-(2-chlorophenyl)-N-(1-methylpropyl)-3-isoquinoline carboxamide; the compounds described in WO 03/030 937 and WO 03/068 753, pyridazino[4,5-b]indole-1-acetamide derivatives of general formula (VII) as described in WO 00/44384.

### 13. Agents for Increasing the Activity of the Sebaceous Glands:

Exemplary are, for example, methyl dehydrojasmonate, hecogenin, hedione and O-linoleyl-6D-glucose, and mixtures thereof.

### 14. Agents for Stimulating the Energy Metabolism of Cells:

The active agent for stimulating the energy metabolism of cells may be selected, for example, from biotin, an extract of Saccharomyces cerevisiae such as Phosphovital® from Sederma, the mixture of sodium, manganese, zinc and magnesium salts of pyrrolidonecarboxylic acid, for instance Physiogenyl® from Solabia, a mixture of zinc, copper and magnesium gluconate, such as Sepitonic M3® from SEPPIC, and mixtures thereof; a beta-glucan derived from Saccharomyces cerevisiae, such as the product marketed by Mibelle AG Biochemistry.

### 15. Tensioning Agents:

The term “tensioning agent” that may be used according to the invention means compounds that provide a tensioning effect, i.e., being able to make the skin taut.

According to the invention, the term “tensioning agent” generally means any polymer that is soluble or dispersible in water at a temperature ranging from 25° C. to 50° C. at a concentration of 7% by weight in water or at the maximum concentration at which a medium of uniform appearance is formed and producing at this concentration of 7% or at this maximum concentration in water a shrinkage of more than 15% in the test described below.

The maximum concentration at which a medium of uniform appearance forms is determined to within ±10% to preferably to within ±5%.

The expression “medium of uniform appearance” means a medium that does not contain any aggregates that are visible to the naked eye.

For the determination of said maximum concentration, the tensioning agent is gradually added to the water with deflocculating stirring at a temperature ranging from 25° C. to 50° C., and the mixture is then stirred for one hour. The mixture thus prepared is then examined after 24 hours to see if it is of uniform appearance (absence of aggregates visible to the naked eye).

The tensioning effect may be characterized by an in vitro shrinkage test.

A homogeneous mixture of the tensioning agent in water, at a concentration of 7% by weight or at the maximum concentration defined above, is prepared beforehand and as described previously.

30 μl of the homogeneous mixture are placed on a rectangular sample (10×40 mm, thus having an initial width L₀ of 10 mm) of elastomer with a modulus of 20 MPa and a thickness of 100 μm.

After drying for 3 hours at 22±3° C. and 40±10% relative humidity RH, the elastomer sample has a shrunken width, noted L₃ₕ, due to the tension exerted by the applied tensioning agent.

The tensioning effect (TE) of said polymer is then quantified in the following manner:

‘TE’=(L₀−L₃ₕ/L₀)×100 as % with L₀=initial width 10 mm and L₃ₕ=width after 3 hours of drying.

The tensioning agent may be selected from:

plant or animal proteins and hydrolysates thereof;

polysaccharides of natural origin;

mixed silicates;

colloidal particles of mineral fillers;

synthetic polymers;

and mixtures thereof.

One skilled in the art will know how to choose, from the chemical categories listed above, the materials corresponding to the tensioning test as described below.

Especially exemplary are:

(a) plant proteins and protein hydrolysates, in particular of corn, rye, wheat, buckwheat, sesame, spelt, pea, bean, lentil, soybean and lupin,

(b) polysaccharides of natural origin, especially (a) polyholosides, for example (i) in the form of starch derived especially from rice, corn, potato, cassava, pea, wheat, oat, etc. or (ii) in the form of carrageenans, alginates, agars, gellans, cellulose polymers and pectins, advantageously as an aqueous dispersion of gel microparticles, and (b) latices constituted of shellac resin, sandarac gum, dammar resins, elemi gums, copal resins, cellulose derivatives, and mixtures thereof,

(c) mixed silicates, especially phyllosilicates and in particular Laponites,

(d) colloidal particles of mineral fillers with a number-average diameter of from 0.1 and 100 nm and preferably from 3 and 30 nm, and selected, for example, from: silica, silica-alumina composites, cerium oxide, zirconium oxide, alumina, calcium carbonate, barium sulfate, calcium sulfate, zinc oxide and titanium dioxide. As silica-alumina composite colloidal particles that may be used in the compositions according to the invention, examples include those marketed by Grace under the trademarks Ludox AM, Ludox AM-X 6021, Ludox HSA and Ludox TMA,

(e) synthetic polymers, such as polyurethane latices or acrylic-silicone latices, in particular those described in EP-1,038,519, such as a polydimethylsiloxane grafted with propylthio(polymethyl acrylate), propylthio(polymethyl methacrylate) and propylthio(polymethacrylic acid), or, alternatively, a polydimethylsiloxane grafted with propylthio(polyisobutyl methacrylate) and propylthio(polymethacrylic acid).

Such grafted silicone polymers are especially marketed by 3M under the trademarks VS 80, VS 70 and LO21.

The tensioning agent will be present in the composition in an amount that is effective for obtaining the desired biological effect according to the invention.

By way of example, the tensioning agent may be included in the composition according to the invention in a content ranging from 0.01% to 30% by weight of active material and preferably from 1% to 30% by weight of active material relative to the total weight of the composition.

The term “active material” is intended to exclude the medium in which the tensioning agent may be dissolved or dispersed in its commercial form, for example in the case of dispersions of colloidal particles.

It is also possible, especially to complement and/or potentiate the effect of tensioning agents, to use agents that increase the expression of mechanoreceptors, such as agents that increase the expression of integrins.

An example that may be mentioned is an extract of rye seed, such as the product marketed by Silab under the trademark Coheliss®.

### 16. Fat-Restructuring Agents:

According to the invention, the term “fat-restructuring agents” means agents capable of stimulating lipogenesis and of promoting adipocyte differentiation, thus making it possible to prevent or slow down the wasting of fat contained in the skin-supporting tissues, also known as “wasting of skin fat”.

The term “skin fat” means the network of fat cells that forms the volumes on which the facial skin rests and is molded.

These agents are useful:

to reduce the loss of skin density and/or the wasting of skin fat, in particular on the cheeks and around the eyes, and/or

to prevent the collapse and/or hollowing of the facial volumes, the loss of consistency of the skin and/or its maintenance, in particular on the cheeks and around the eyes, and/or

to improve the underlying volumes of the skin of the face and/or the neck, in particular on the cheeks, the oval of the face and around the eyes, and/or

to improve the density, springiness and maintenance of the skin, in particular on the cheeks, the oval of the face and around the eyes, and/or

to remodel the facial features, in particular the oval of the face.

Examples of fat-restructuring agents that may especially be mentioned include an extract of black tea, such as the extract of fermented black tea marketed by Sederma under the trademark Kombuchka®, and an extract of Artemisia abrotanum, such as the product marketed by Silab under the trademark Pulpactyl®.

### 17. Slimming Agents:

Slimming (lipolytic) agents that may especially be mentioned include caffeine, theophylline and its derivatives, theobromine, sericosine, asiatic acid, acefylline, aminophylline, chloroethyltheophylline, diprofylline, diniprophylline, etamiphylline and its derivatives, etofylline and proxyphylline; extracts of tea, of coffee, of guarana, of maté, of cola (Cola nitida) and especially the dry extract of guarana fruit (Paulina sorbilis) containing 8% to 10% caffeine; extracts of climbing ivy (Hedera helix), of arnica (Arnica montana L), of rosemary (Rosmarinus officinalis N), of marigold (Calendula officinalis), of sage (Salvia officinalis L), of ginseng (Panax ginseng), of St. John's wort (Hypericum perforatum), of butcher's broom (Ruscus aculeatus L), of meadowsweet (Filipendula ulmaria L), of orthosiphon (Orthosiphon stamincus Benth), of birch (Betula alba), of pumpwood and of argan tree, extracts of ginkgo biloba, extracts of horsetail, extracts of escin, extracts of cangzhu, extracts of Chrysanthellum indicum, extracts of diosgenin-rich Dioscorea plants or pure diosgenin or hecogenin and derivatives thereof, extracts of Ballota, extracts of Guioa, of Davaffia, of Terminalia, of Barringtonia, of Trema or of Antirobia, the extract of bitter orange pips; an extract of husks of cocoa beans (Theobroma cacao) such as the product marketed by Solabia under the trademark Caobromine®.

### 18. Agents for Promoting the Cutaneous Capillary Circulation:

The active agent acting on the cutaneous capillary circulation may be used for preventing dulling of the complexion and/or to improve the appearance of the area around the eyes, in particular to reduce the shadows around the eyes. It may be selected, for example, from an extract of maritime pine bark, for instance Pycnogenol® from Biolandes, manganese gluconate (Givobio GMn® from SEPPIC), an extract of Ammi visnaga such as Visnadine from Indena, extract of lupin (Eclaline® from Silab), the protein coupling of hydrolysed wheat/palmitic acid with palmitic acid, such as Epaline 100 from Laboratoires Carilène, the extract of bitter orange blossom (Remoduline® from Silab), vitamin P and derivatives thereof, for instance methyl-4 esculetol sodium monoethanoate marketed under the trademark Permethol® by Sephytal, extracts of Ruscus, of common horse chestnut, of ivy, of ginseng and of melilot, caffeine, nicotinate and derivatives thereof, lysine and derivatives thereof, for instance Asparlyne® from Solabia, an extract of black tea such as Kombuchka from Sederma; rutin salts; an extract of the alga Corallina officinalis, such as the product marketed by Codif; and mixtures thereof.

As preferred agents for promoting the cuteneous capillary circulation, mention will be made of caffeine, an extract of bitter orange blossom, an extract of black tea, rutin salts and an extract of the alga Corallina officinalis.

### 19. Calmatives or Anti-Irritants:

The term “calmative” means a compound that can reduce the sensation of stinging, itching or tautness of the skin.

As calmatives that may be included in the compositions according to the invention, exemplary are: procyanidol oligomers, vitamins E, B5 and B3, caffeine and derivatives thereof, pentacyclic triterpenes and plant extracts containing them, β-glycyrrhetinic acid and salts or derivatives thereof (stearyl glycyrrhetate, 3-stearoyloxyglycyrrhetic acid or glycyrrhetinic acid monoglucuronide) and also plants containing them (e.g., Glycyrrhiza glabra), oleanolic acid and salts thereof, ursolic acid and salts thereof, boswellic acid and salts thereof, betulinic acid and salts thereof, an extract of Paeonia suffruticosa and/or lactiflora, an extract of Laminaria saccharina, extracts of Centella asiatica, Canola oil, bisabolol, the phosphoric diester of vitamin E and C, for instance Sepivital EPC® from SEPPIC, camomile extracts, allantoin, omega-3 unsaturated oils such as musk rose oil, blackcurrant oil, Ecchium oil, fish oil or beauty-leaf oil, plankton extracts, capryloyl glycine, a mixture of water lily blossom extract and of palmitoylproline, such as the product marketed under the trademark Seppicalm VG® by SEPPIC, an extract of Boswellia serrata, an extract of Centipeda cunninghami, such as the product marketed under the trademark Cehami PF® by TRI-K Industries, an extract of sunflower seeds, in particular Hélioxine® from Silab, an extract of Linum usitatissimum seeds, for instance Sensiline® from Silab, tocotrienols, piperonal, an extract of Epilobium angustifolium, such as the product marketed under the trademark Canadian Willowherb Extract by Fytokem Products, Aloe vera, phytosterols, cornflower water, rose water, an extract of mint, in particular of mint leaves, for instance Calmiskin® from Silab, aniseed derivatives, filamentous bacteria, for instance Vitreoscilla filiformis as described in EP-761,204 and marketed by Chimex under the trademark Mexoryl SBG®, an extract of rose petals, for instance Rose Flower Herbasol® extract from the company Cosmetochem, shea butter, a mixture of the waxy fraction of barley seeds obtained by supercritical CO₂, of shea butter and of argan oil, for instance Stimu-tex AS® from Pentapharm, alkaline-earth metal salts, especially of strontium, a fermented extract of Alteromonas marketed under the trademark Abyssine® by Atrium Biotechnologies; spring water from the Vichy basin, such as waters originating from the Célestin, Chomel, Grande-Grille, Hôpital, Lucas and Parc sources, and preferably water from the Lucas source; an extract of Eperua falcata bark, such as the product marketed by Cognis under the trademark Eperuline®; an extract of Paeonia suffruticosa root, such as the product marketed by Ichimaru Pharcos under the trademark Botanpi Liquid B®; and mixtures thereof.

As preferred calmatives according to the invention, use will be made of:

β-glycyrrhetinic acid and salts or derivatives thereof (stearyl glycyrrhetate, 3-stearoyloxyglycyrrhetic acid or glycyrrhetinic acid monoglucuronide) and also plants containing them (e.g., Glycyrrhiza glabra); ursolic acid and salts thereof; extracts of Centella asiatica, Canola oil, bisabolol; camomile extracts, allantoin; a mixture of extract of water lily blossom and of palmitoylproline, such as the product marketed under the trademark Seppicalm VG® by SEPPIC; Aloe vera, rose water, extract of mint, in particular of mint leaves, such as Calmiskin® from Silab, filamentous bacteria such as Vitreoscilla filiformis as described in EP-761,204 and marketed by Chimex under the trademark Mexoryl SBG®, an extract of rose petals such as Rose Flower Herbasol® extract from the company Cosmetochem, shea butter, a fermented extract of Alteromonas marketed under the trademark Abyssine® by Atrium Biotechnologies; spring water from the Vichy basin, such as waters originating from the Célestin, Chomel, Grande-Grille, Hôpital, Lucas and Parc sources, and preferably water from the Lucas source; an extract of Eperua falcata bark, such as the product marketed by Cognis under the trademark Eperuline®; an extract of Paeonia suffruticosa root, such as the product marketed by Ichimaru Pharcos under the trademark Botanpi Liquid B®; and mixtures thereof.

### 20. Sebo-Regulating or Anti-Seborrhoeic Agents:

The term “sebo-regulating or anti-seborrhoeic agents” especially means agents capable of regulating the activity of the sebaceous glands.

Especially exemplary are:

retinoic acid, benzoyl peroxide, sulfur, vitamin B6 (or pyridoxine), selenium chloride and sea fennel;

mixtures of extract of cinnamon, of tea and of octanoylglycine such as Sepicontrol A5 TEA® from SEPPIC;

the mixture of cinnamon, sarcosine and octanoylglycine marketed especially by SEPPIC under the trademark Sepicontrol A5®;

zinc salts such as zinc gluconate, zinc pyrrolidonecarboxylate (or zinc pidolate), zinc lactate, zinc aspartate, zinc carboxylate, zinc salicylate and zinc cysteate;

copper derivatives and in particular copper pidolate such as Cuivridone® from Solabia;

extracts of plants of the species Arnica montana, Cinchona succirubra, Eugenia caryophyllata, Humulus lupulus, Hypericum perforatum, Mentha piperita, Rosmarinus officinalis, Salvia oficinalis and Thymus vulgaris, all marketed, for example, by Maruzen;

extracts of meadowsweet (Spiraea ulmaria), such as the product marketed under the trademark Sebonormine® by Silab;

extracts of the alga Laminaria saccharina, such as the product marketed under the trademark Phlorogine® by Biotechmarine;

mixtures of extracts of salad burnet root (Sanguisorba officinalis/Poterium officinale), of ginger rhizomes (Zingiber officinalis) and of cinnamon bark (Cinnamomum cassia), such as the product marketed under the trademark Sebustop® by Solabia;

linseed extracts, such as the product marketed under the trademark Linumine® by Lucas Meyer;

Phellodendron extracts, such as those marketed under the trademark Phellodendron extract BG by Maruzen or Oubaku liquid B by Ichimaru Pharcos;

mixtures of argan oil, of Serenoa serrulata (saw palmetto) extract and of sesame seed extract, such as the product marketed under the trademark Regu SEB® by Pentapharm;

mixtures of extracts of willowherb, of Terminalia chebula, of nasturtium and of bioavailable zinc (microalgae), such as the product marketed under the trademark Seborilys® by Green Tech;

extracts of Pygeum afrianum, such as the product marketed under the trademark Pygeum afrianum sterolic lipid extract by Euromed;

extracts of Serenoa serrulata, such as the products marketed under the trademark Viapure Sabal by Actives International or those marketed by Euromed;

mixtures of extracts of plantain, of Berberis aquifolium and of sodium salicylate, such as the product marketed under the trademark Seboclear® by Rahn;

clove extract, such as the product marketed under the trademark Clove extract powder by Maruzen;

argan oil, such as the product marketed under the trademark Lipofructyl® by Laboratoires Sérobiologiques,

lactic protein filtrates, such as the product marketed under the trademark Normaseb® by Sederma;

extracts of the alga Laminaria, such as the product marketed under the trademark Laminarghane® by Biotechmarine;

oligosaccharides of the alga Laminaria digitata, such as the product marketed under the trademark Phycosaccharide AC by Cod if;

sugar cane extracts, such as the product marketed under the trademark Policosanol® by Sabinsa;

sulfonated shale oil, such as the product marketed under the trademark Ichthyol Pale® by Ichthyol;

European meadowsweet (Spiraea ulmaria) extracts, such as the product marketed under the trademark Cytobiol® Ulmaire by Libiol;

sebacic acid, especially marketed in the form of a sodium polyacrylate gel under the trademark Sebosoft® by Sederma;

glucomannans extracted from konjac tuber and modified with alkylsulfonate chains, such as the product marketed under the trademark Biopol Beta by Arch Chemical;

extracts of Sophora angustifolia, such as those marketed under the trademark Sophora powder or Sophora extract by Bioland;

extracts of Cinchona succirubra bark, such as the product marketed under the trademark Red Bark HS by Alban Muller;

extracts of Quillaja saponaria, such as the product marketed under the trademark Panama wood HS by Alban Muller;

glycine grafted onto an undecylenic chain, such as the product marketed under the trademark Lipacide UG OR by SEPPIC;

the mixture of oleanolic acid and of nordihydroguaiaretic acid, such as the product marketed in the form of a gel under the trademark AC.Net by Sederma;

phthalimidoperoxyhexanoic acid;

tri(C₁₂-C₁₃)alkyl citrate marketed under the trademark Cosmacol® ECI by Sasol; tri(C₁₄-C₁₅)alkyl citrate marketed under the trademark Cosmacol® ECL by Sasol;

### 10-hydroxydecanoic acid, and especially mixtures of 10-hydroxydecanoic acid, of sebacic acid and of 1,10-decanediol, such as the product marketed under the trademark Acnacidol® BG by Vincience; and

mixtures thereof.

Preferred anti-seborrhoeic active agents that are exemplary include:

benzoyl peroxide and vitamin B6 (or pyridoxine),

zinc salts such as zinc gluconate, zinc pyrrolidonecarboxylate (or zinc pidolate), zinc lactate, zinc aspartate, zinc carboxylate, zinc salicylate and zinc cysteate;

meadowsweet (Spiraea ulmaria) extracts, such as the product marketed under the trademark Sebonormine® by Silab;

extracts of the alga Laminaria saccharina, such as the product marketed under the trademark Phlorogine® by Biotechmarine;

mixtures of extracts of salad burnet root (Sanguisorba officinalis/Poterium officinale), of ginger rhizomes (Zingiber officinalis) and of cinnamon bark (Cinnamomum cassia), such as the product marketed under the trademark Sebustop® by Solabia;

clove extract, such as the product marketed under the trademark Clove extract powder by Maruzen;

lactic protein filtrates, such as the product marketed under the trademark Normaseb® by Sederma;

European meadowsweet (Spiraea ulmaria) extracts, such as the product marketed under the trademark Cytobiol® Ulmaire by Libiol;

sebacic acid, especially marketed in the form of a sodium polyacrylate gel under the trademark Sebosoft® by Sederma;

glycine grafted onto an undecylenic chain, such as the product marketed under the trademark Lipacide UG OR by SEPPIC;

tri(C₁₂-C₁₃)alkyl citrate marketed under the trademark Cosmacol® ECI by Sasol; tri(C₁₄-C₁₅)alkyl citrate marketed under the trademark Cosmacol® ECL by Sasol;

10-hydroxydecanoic acid, and especially mixtures of 10-hydroxydecanoic acid, of sebacic acid and of 1,10-decanediol, such as the product marketed under the trademark Acnacidol® BG by Vincience; and

mixtures thereof.

Preferentially, the anti-seborrhoeic active agent is selected from:

zinc salts such as zinc gluconate, zinc pyrrolidonecarboxylate (or zinc pidolate), zinc lactate, zinc aspartate, zinc carboxylate, zinc salicylate and zinc cysteate; and preferably zinc pyrrolidonecarboxylate (or zinc pidolate) or zinc salicylate;

clove extract, such as the product marketed under the trademark Clove extract powder by Maruzen;

glycine grafted onto an undecylenic chain, such as the product marketed under the trademark Lipacide UG OR by SEPPIC;

tri(C₁₂-C₁₃)alkyl citrate marketed under the trademark Cosmacol® ECI by Sasol; tri(C₁₄-C₁₅)alkyl citrate marketed under the trademark Cosmacol® ECL by Sasol;

and mixtures thereof.

The anti-seborrhoeic active agent is, for example, present in a content ranging from 0.1% to 10% by weight, preferably from 0.1% to 5% by weight and preferentially from 0.5% to 3% by weight relative to the total weight of the composition.

### 21. Astringents:

According to the invention, the term “astringents” means agents for combating the dilation of the sebaceous follicles.

As astringents that may be included in the compositions according to the invention, exemplary are extracts of mushroom pulp (Polyporus officinalis), for instance Laricyl LS8865® from Cognis, extracts of Terminalia catappa and Sambucus nigra, for instance Phytofirm LS9120® from Cognis, extracts of gall nut, for instance Tanlex VE® from Ichimaru Pharcos, aluminum hydroxychloride, centella extracts (e.g., Plantactiv centella from Cognis), dicetyl dimethylammonium chloride, for instance Varisoft 432 CG® from Degussa, common horse-chestnut extracts, mallow extracts, witch-hazel extracts, sweet almond extracts, marshmallow root extracts and linseed extracts, for instance Almondermin LS 3380® from Cognis, burdock extracts, nettle extracts, birch extracts, horsetail extracts, camomile extracts, for instance those marketed under the trademark Extrapone 9 Special® by Symrise, skullcap extracts, European meadowsweet extracts (for example Cytobiol Ulmaire from Libiol), a mixture of extracts of white ginger, of horsetail, of nettle, of rosemary and of yucca, for instance Herb extract B1348® from Bell Flavors & Fragrances, extracts of acacia, of elm, of white willow, of cinnamon, of birch and of meadowsweet, Panama sapogenins, zinc phenolsulfonate from Interchemical, extracts of gentian, of cucumber and of walnut, the mixture of extracts of Ratanhia, of grapefruit, of gumweed and of oak gall, for instance Epilami® from Alban Muller.

As preferred astringents according to the invention, use will be made of skullcap extracts, European meadowsweet extracts, meadowsweet extracts, gentian extracts and burdock extracts, and mixtures thereof.

### 22. Cicatrizing Agents:

Examples of cicatrizing agents that may especially be mentioned include:

allantoin, urea, certain amino acids, for instance hydroxyproline, arginine, and serine, and also extracts of white 111y (for instance Phytélène Lys 37EG 16295 from Indena), a yeast extract, for instance the cicatrizing agent LS LO/7225B from Laboratoires Sérobiologiques), tamanu oil, extract of Saccharomyces cerevisiae, for instance Biodynes® TRF® from Arch Chemical, oat extracts, chitosan and derivatives, for instance chitosan glutamate, carrot extracts, artemia extract, for instance GP4G® from Vincience, sodium acexamate, lavandin extracts, propolis extracts, ximeninic acid and salts thereof, rose hip oil, marigold extracts, for instance Souci Ami® Liposolible from Alban Muller, horsetail extracts, lemon peel extracts, for instance Herbasol® citron from Cosmetochem, helichrysum extracts, common yarrow extracts and folic acid.

As preferred cicatrizing agents according to the invention, use will be made of arginine, serine, folic acid, tamanu oil, sodium acexamate, horsetail extracts and helichrysum extracts, and mixtures thereof.

### 23. Anti-Inflammatory Agents:

As particular anti-inflammatory agents that may be used according to the invention, exemplary are cortisone, hydrocortisone, indomethacin, betamethasone, azelaic acid, acetaminophen, diclofenac, clobetasol propionate, folic acid; an extract of Eperua falcata bark, such as the product marketed by Cognis under the trademark Eperuline®; an extract of Paeonia suffruticosa root, such as the product marketed by Ichimaru Pharcos under the trademark Botanpi Liquid B®; and mixtures thereof.

Preferred anti-inflammatory agents that will be mentioned are azelaic acid, folic acid, an extract of Eperua falcata bark, such as the product marketed by Cognis under the trademark Eperuline®; an extract of Paeonia suffruticosa root, such as the product marketed by Ichimaru Pharcos under the trademark Botanpi Liquid B®; and mixtures thereof.

24. Anti-acne agents:

In one advantageous aspect of the invention, the composition may also comprise at least one anti-acne active agent.

The term “anti-acne active agent” especially means any active agent that has effects on the specific flora of greasy skin, for instance Propionibacterium acnes (P. acnes).

These effects may be bactericidal.

Antibactericidal active agents that may especially be mentioned include:

active agents and preservatives with antimicrobial activity mentioned in DE-103,24,567, which is incorporated into the present invention by reference,

Asiatic acid,

the monoethanolamine salt of 1-hydroxy-4-methyl 6-trimethylpentyl-2-pyridone (INCI name: piroctone olamine), marketed especially under the trademark Octopirox® by Clariant;

citronellic acid, perillic acid (or 4-isopropenylcyclohex-1-enecarboxylic acid),

glyceryl 2-ethylhexyl ether (INCI name: ethylhexylglycerine), for example marketed under the trademark Sensiva SC 50® by Shulke & Mayr,

glyceryl caprylate/caprate, for example marketed under the trademark Capmul MCM® by Abitec;

sodium calcium phosphosilicate, especially marketed under the trademarks Bioactive Glasspowder® and Actysse Premier BG® by Schott Glass;

silver-based particles, for example those marketed under the trademark Metashine ME 2025 PS® by Nippon Sheet Glass;

hop cone extract (Humulus lupulus) obtained by supercritical CO₂ extraction, such as the product marketed under the trademark HOP CO2-TO Extract® by Flavex Naturextrakte,

St.-John's Wort extract obtained by supercritical CO₂ extraction, such as the product marketed under the trademark St.-John's Wort CO2-TO extract® by Flavex Naturextrakte,

the mixture of extracts of roots of Scutellaria baicalensis, of Paeonia suffruticosa and Glycyrrhiza glabra, such as the product marketed under the trademark BMB-CF® by Naturogin,

argan tree extract, for instance Argapure LS9710® from Cognis;

bearberry leaf extracts, for instance the product marketed under the trademark Melfade-J by Pentapharm;

10-hydroxy-2-decanoic acid such as Acnacidol P® from Vincience, sodium ursolate, azelaic acid, diiodomethyl p-tolyl sulfone such as Amical Flowable® from Angus, malachite powder, zinc oxide such as Zincare® from Elementis GMBH, octadecenedioic acid such as Arlatone dioic DCA® from Uniqema; ellagic acid; 2,4,4′-trichloro-2′-hydroxydiphenyl ether (or triclosan), 1-(3′,4′-dichlorophenyl)-3-(4′-chlorophenyl)urea (or triclocarban), 3,4,4′-trichlorocarbanilide, 3′,4′,5′-trichlorosalicylanilide, phenoxyethanol, phenoxypropanol, phenoxyisopropanol, hexamidine isethionate, metronidazole and salts thereof, miconazole and salts thereof, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and salts thereof, benzoyl peroxide, 3-hydroxybenzoic acid, 4-hydroxybenzoic acid, phytic acid, N-acetyl-L-cysteine, lipoic acid, azelaic acid and salts thereof, arachidonic acid, resorcinol, 3,4,4′-trichlorocarbanalide, octoxyglycerine or octoglycerine, octanoylglycine such as Lipacid C8G® from SEPPIC, caprylyl glycol, 10-hydroxy-2-decanoic acid, dichlorophenylimidazoldioxolane and derivatives thereof described in WO 93/18743, iodopropynyl butylcarbamate, 3,7,11-trimethyldodeca-2,5,10-trienol or farnesol, phytosphingosines; quaternary ammonium salts, for instance cetyltrimethylammonium salts and cetylpyridinium salts, and

mixtures thereof.

Mention may also be made of certain surfactants with an antimicrobial effect, for instance sodium cocoamphoacetate or disodium diacetate such as Miranol C2M Conc. NP, betaines, for instance the cocoyl betaine Genagen KB from Clariant, sodium lauryl ether sulfate, for instance Emal 270 D from Kao, decyl glucoside, for instance Plantacare 2000 UP, branched C₁₂₋₁₃ dialkyl malates, for instance Cosmacol EMI, propylene glycol monoesters, for instance propylene glycol monolaurate, monocaprylate or monocaprate, lauryldimethylamine betaine, for instance Empigen BB/LS, and also polyquaternary ammoniums such as Quaternium-24 or Bardac 2050 from Lonza and those described in FR-0,108,283, and mixtures thereof.

As preferred antimicrobial agents, an agent selected from octoglycerine or octoxyglycerine, and 10-hydroxy-2-decanoic acid, and mixtures thereof, will be used in the compositions of the invention.

Other additional anti-acne active agents may be added to the abovementioned anti-acne active agents.

Especially exemplary are active agents with bacterial anti-adhesion effects or agents that act on the biofilm of bacteria to prevent them from multiplying.

As agents for preventing and/or reducing the adhesion of microorganisms, especially exemplary are: phytanetriol and derivatives thereof as described in EP-1,529,523, plant oils such as wheatgerm oil, calendula oil, castor oil, olive oil, avocado oil, sweet almond oil, groundnut oil, jojoba oil, sesame seed oil, apricot kernel oil, sunflower oil and macadamia oil, described in EP-1,133,979, or certain surfactants such as disodium cocoamphodiacetate, oxyethylenated (7 EO) glyceryl cocoate, 18-hexadecenyl succinate, octoxyglyceryl palmitate, octoxyglyceryl behenate, dioctyl adipate, PPG-15 stearyl ether, and the branched C₁₂-C₁₃ dialkyl tartrates described in EP-1,129,694, and mixtures thereof.

In particular with regard to the propagation of P. acnes, or as active agents that act on the biofilm of bacteria to prevent them from proliferating, exemplary are pentylene glycol, Nylon-66 (polyamide 66 fibers), rice bran oil, polyvinyl alcohol such as Celvol 540 PV alcohol® from Celanese Chemical, rapeseed oil such as Akorex L® from Karlshamns, and fructose derivatives, and mixtures thereof.

The antiacne active agent may be present in a content ranging from 0.01% to 10% by weight, preferably from 0.05% to 5% by weight, relative to the total weight of the composition.

According to the nature and/or the solubility of the above-mentioned active agents, those skilled in the art will know how to select the most suitable embodiment according to the invention.

The cosmetic and/or dermatological active agents will be present in one of the compositions according to the invention in a content ranging from 0.001% to 20% by weight, relative to the total weight of the composition, preferably from 0.01% to 10%, even more preferentially from 0.5% to 5%, and more preferably from 0.1% to 1% by weight, relative to the total weight of the composition.

For “peeling” applications, the contents of cosmetic and/or dermatological active agents may range from 1% to 50% by weight, relative to the total weight of the composition, preferably from 1% to 30% by weight, relative to the total weight of the composition.

Peelings are a well-known means for improving the appearance and/or the texture of the skin and/or of the scalp, especially for improving the radiance and homogeneity of the complexion and/or for reducing the visible and/or tactile irregularities of the skin, and in particular for improving the surface appearance of the skin, for attenuating actinic lentigo, acne or chicken pox marks, and also for preventing, attenuating or combatting the signs of skin aging, and especially for smoothing out irregularities in the texture of the skin, such as wrinkles and fine lines.

They have the effect of removing a surface part of the skin to be treated (epidermis and possibly the upper layer of the dermis), via chemical methods.

In order to further illustrate the present invention and the advantages thereof, the following specific examples are given, it being understood that same are intended only as illustrative and in nowise limitative. In said examples to follow, all parts and percentages are given by weight, unless otherwise indicated.

### Example 1

### Oil-in-Water Emulsion

| Chemical name | % by weight | Phase |
|---|---|---|
| MIXTURE OF OXYETHYLENATED | 10 | A |
| OXYPROPYLENATED (18 EO/18 | | |
| PO) POLYDIMETHYLSILOXANE, OF | | |
| CYCLOPENTADIMETHYLSILOXANE | | |
| AND OF WATER (10/88/2)* | | |
| (DOW CORNING 5225C | | |
| FORMULATION AID) | | |
| CYCLOPENTADIMETHYLSILOXANE | 12.5 | |
| (DOW CORNING 245 FLUID) | | |
| TOCOPHEROL | 0.1 | |
| SODIUM CHLORIDE | 2 | B |
| WATER | 15 | |
| BETA-CAROTENE AT 30% | 0.432 | C |
| (CAROTENE-DISPERSION | | |
| NATURAL 30% L-OS E-160A) | | |
| EXTRACT OF THE ALGA | 0.084 | |
| HAEMATOCOCCUS PLUVIALIS | | |
| CONTAINING 6% ASTAXANTHIN | | |
| AS A DISPERSION IN | | |
| TRIGLYCERIDES (ASTA TROL-X) | | |
| SODIUM SALT OF COPPER- | 0.084 | |
| CONTAINING CHLOROPHYLL AT | | |
| 15% (COPPER CHLOROPHYLL | | |
| 15% L-OS) | | |
| DIPROPYLENE GLYCOL | 10 | D |
| PROPYLENE GLYCOL | 25.5 | |
| WATER | 19.3 | |
| DENATURED ALCOHOL | 5 | E |

Composition of the Dye Mixture with Respect to Active Material:

| | % by weight active | |
|---|---|---|
| | material in the | Relative proportion in |
| Dye | formulation | the dye mixture |
| BETA-CAROTENE | 0.1296 | 88.163 |
| ASTAXANTHIN | 0.00504 | 3.42856 |
| CHLOROPHYLL | 0.0126 | 8.5714 |

This formulation was tested in vivo on a panel of about ten women corresponding to light skin types having a flesh tone ranging from Ito III according to the Fitzpatrick scale. A homogeneous color and an immediate “healthy complexion” effect are observed, without the natural appearance of the skin being masked.

### Example 2

### Water-in-Oil Emulsion

| Chemical name | % by weight | Phase |
|---|---|---|
| MIXTURE OF OXYETHYLENATED | 10 | A |
| OXYPROPYLENATED (18 EO/18 | | |
| PO) POLYDIMETHYLSILOXANE, OF | | |
| CYCLOPENTADIMETHYLSILOXANE | | |
| AND OF WATER (10/88/2)* | | |
| (DOW CORNING 5225C | | |
| FORMULATION AID) | | |
| CYCLOPENTADIMETHYLSILOXANE | 12.5 | |
| (DOW CORNING 245 FLUID) | | |
| TOCOPHEROL | 0.1 | |
| SODIUM CHLORIDE | 2 | B |
| WATER | 15 | |
| BETA-CAROTENE AT 30% | 1.44 | C |
| (CAROTENE-DISPERSION | | |
| NATURAL 30% L-OS E-160A) | | |
| EXTRACT OF THE ALGA | 0.28 | |
| HAEMATOCOCCUS PLUVIALIS | | |
| CONTAINING 6% ASTAXANTHIN | | |
| AS A DISPERSION IN | | |
| TRIGLYCERIDES (ASTA TROL-X) | | |
| SODIUM SALT OF COPPER- | 0.28 | |
| CONTAINING CHLOROPHYLL AT | | |
| 15% (COPPER CHLOROPHYLL | | |
| 15% L-OS) | | |
| DIPROPYLENE GLYCOL | 10 | D |
| PROPYLENE GLYCOL | 25.5 | |
| WATER | 17.9 | |
| DENATURED ALCOHOL | 5 | E |

Composition of the Lipophilic Dye Mixture with Respect to Active Material:

| | % by weight active | |
|---|---|---|
| | material in the | Relative proportion in |
| Dye | formulation | the dye mixture |
| BETA-CAROTENE | 0.432 | 88.0195 |
| ASTAXANTHIN | 0.0168 | 3.42298 |
| CHLOROPHYLL | 0.042 | 8.55745 |

This formulation was tested in vivo on a panel of about ten women corresponding to dark skin types having a flesh tone ranging from IV to VI according to the Fitzpatrick scale. A homogeneous color and an immediate “healthy complexion” effect are observed, without the natural appearance of the skin being masked.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method for artificially coloring the skin without masking the natural appearance thereof, which comprises topically applying thereon a thus effective amount of a composition comprising:
a) at least one carotene compound,
b) at least one xanthophyll compound, and
c) at least one lipophilic green dye, formulated into a topically applicable, physiologically acceptable medium therefor.

2. The artificial skin coloration method as defined by claim 1, in which the at least one carotene compound comprises β-carotene.

3. The artificial skin coloration method as defined by claim 1, in which the at least one xanthophyll compound comprises astaxanthin.

4. The artificial skin coloration method as defined by claim 1, in which the at least one lipophilic green dye is selected from among quinizarin and the chlorophylls.

5. The artificial skin coloration method as defined by claim 4, in which the at least one green dye comprises a chlorophyll in the form of a copper complex dispersion in an oil.

6. The artificial skin coloration method as defined by claim 1, in which the composition comprises a mixture of lipophilic dyes in an amount of from 1% to 10% by weight of active material of green dye, relative to the total weight of the mixture of lipophilic dyes.

7. The artificial skin coloration method as defined by claim 1, in which a mixture of lipophilic dyes is present in the composition in concentrations ranging from 0.1% to 25%, relative to the total weight of the composition.

8. The artificial skin coloration method as defined by claim 7, in which the mixture of lipophilic dyes comprises:
a) from 70% to 90% by weight of active material of carotene compound;
b) from 1% to 20% by weight of active material of xanthophyll compound;
c) from 1% to 10% by weight of active material of green dye, the amounts being defined relative to the total weight of the mixture and the sum of the amounts being equal to 100%.

9. The artificial skin coloration method as defined by claim 1, comprising:
1) a coloring of the skin with a composition (A) based on a mixture of lipophilic dyes, followed or preceded by;
2) a coloring of the skin obtained with a composition (B) comprising, in a physiologically acceptable medium, an effective amount of at least one dye precursor selected from compounds containing at least one aromatic ring having at least two hydroxyl (OH) groups borne by two consecutive carbon atoms of the aromatic ring and an effective amount of a catalytic system comprising a first constituent (B₁) selected from the salts and oxides of Mn(II) and/or Zn(II), and mixtures thereof, and a second constituent (B₂) selected from alkali metal hydrogen carbonates, alkaline-earth metal hydrogen carbonates, and mixtures thereof, the proportions of the first constituent and the second constituent being such that:
$$\frac{[ Mn ( II ) ]}{[ {HCO}_{3} ]} \leq 1 with [ Mn ( II ) ] \neq 0$$
$$\frac{[ Zn ( II ) ]}{[ {HCO}_{3} ]} \leq 1 with [ Zn ( II ) ] \neq 0$$
$$\frac{⌊ Mn ( II ) + Zn ( II ) ⌋}{[ {HCO}_{3} ]} \leq 1 with [ Mn ( II ) ] and [ Zn ( II ) ] \neq 0$$
wherein:
[Mn(II)], [Zn(II)] and [HCO₃] represent, respectively, the molar concentrations of Mn(II), Zn(II) and HCO₃ in the composition.

10. The artificial skin coloration method as defined by claim 1, in which the composition comprises a mixture of lipophilic dyes, at least one self-tanning agent and/or at least one stabilizer and/or at least one photoprotective agent and/or at least one additional dye.

11. A mixture of lipophilic dyes, comprising:
a) at least one carotene compound,
b) astaxanthin, and
c) at least one lipophilic green dye.

12. The lipophilic dye mixture as defined by claim 11, in which the at least one carotene compound comprises a dispersion of β-carotene in an oil.

13. The lipophilic dye mixture as defined by claim 11, in which the at least one green dye comprises a chlorophyll compound in the form of a copper complex dispersion in an oil.

14. The lipophilic dye mixture as defined by claim 11, comprising a mixture of lipophilic dyes in an amount of from 1% to 10% by weight of active material of green dye relative to the total weight of the mixture of lipophilic dyes.

15. A composition for artificially coloring the skin, comprising, formulated into a physiologically acceptable medium, at least one mixture of lipophilic dyes as defined by claim 11.

16. The composition as defined by claim 15, in which the mixture of lipophilic dyes is present in concentrations ranging from 0.1% to 25% by weight, relative to the total weight of the composition.

17. The composition as defined by claim 15, in which the composition based on the mixture of lipophilic dyes also comprises at least one self-tanning agent and/or at least one stabilizer and/or at least one photoprotective agent and/or at least one additional dye.

18. The artificial skin coloration method as defined by claim 2, in which the at least one carotene compound comprises a dispersion of β-carotene in an oil.

19. The artificial skin coloration method as defined by claim 3, in which the at least one xanthophyll compound comprises a dispersion of astaxanthin in an oil.
